(19) United States
(12) Patent Application Publication
(10) Pub. No.: US 2026/0284399 A1
(43) Pub. Date: Sep. 24, 2026

(54) MULTI-MODAL NEUROMODULATION

(71) Applicant: Case Western Reserve University, Cleveland, OH (US)

(72) Inventors: Tina VRABEC, Cleveland, OH (US); Shane BENDER, Cleveland, OH (US); Kevin KILGORE, Cleveland, OH (US); Niloy BHADRA, Cleveland, OH (US); David GREEN, Cleveland, OH (US)

(21) Appl. No.: 19/474,906

(22) PCT Filed: Apr. 12, 2024
(86) PCT No.: PCT/US2024/024362

§ 371 Date: Oct. 13, 2025

Related U.S. Application Data
(60) Provisional application No. 63/496,106, filed on Apr. 14, 2023.

(51) Int. Cl. A61N 1/36; G16H 20/30

(52) U.S. Cl. CPC A61N 1/36114; A61N 1/36135; A61N 1/36189; G16H 20/30

(57) ABSTRACT

Multi-modal neuromodulation applied to an autonomic nerve using a multicontact electrode can upregulate a portion of the autonomic nervous system and downregulate another portion of the autonomic nervous system to affect a physiological condition. A generator can generator and provide a neuromodulation signal and another neuromodulation signal, each having at least one parameter, to the electrode. The neuromodulation signal can be applied at a time and the other neuromodulation signal can be applied at another time to balance sympathetic and parasympathetic activity. A sensor can record a physiological parameter related to physiological condition and a controller can receive the recoding and can adjust which neuromodulation signal is applied and/or at least one of the parameters of the neuromodulation signal or the other neuromodulation signal based on the recording of the physiological parameter.

### CROSS REFERENCE TO RELATED APPLICATIONS

This application claims the benefit of U.S. Provisional Application Ser. No. 63/496,106, filed 14 Apr. 2023, entitled “BI-MODAL NEUROMODULATION”. The entirety of this application is incorporated by reference for all purposes.

### GOVERNMENT FUNDING

This invention was made with government support under R01-HL150136 and U41 NS129436 awarded by the National Institutes of Health. The government has certain rights in the invention.

### TECHNICAL FIELD

This disclosure relates generally to neuromodulation and more specifically to multimodal neuromodulation that up-regulates at least a portion of a patient's autonomic nervous system and down-regulates at least another portion of the patient's autonomic nervous system to affect at least one physiological condition.

### BACKGROUND

Cardiovascular diseases are continually progressive conditions that are often rapidly changeable and difficult to treat. If left untreated or incorrectly treated, such cardiovascular diseases can severely, and even fatally, affect patients. Initial treatments can include the prescription of pharmaceuticals, which are easy to take but can have dangerous side effects and/or contraindications. As the diseases progress and the patients' conditions worsen, more invasive treatment options often become necessary. Examples of these more invasive treatment options that can provide electrical stimulation directly to the heart muscles include pacemakers and implanted defibrillators. However, such options tend to be extremely invasive with stimulation leads placed directly against the heart wall and can have a number of negative side effects. There exists a need for a less invasive and safer method to effectively treat and/or control progressive cardiovascular diseases.

### SUMMARY

Described herein are systems and methods for multimodal neuromodulation of one or more neural structures of a patient suffering a physiological condition. The multimodal neuromodulation can apply at least one stimulating electrical signal and at least one blocking electrical signal to control abnormal changes in at least one physiological parameter related to the physiological condition the patient suffers from. It should be noted that in some instances, sequential application of the at least one stimulating electrical signal and the at least one blocking electrical signal is necessary to regulate activity in a nerve to control the abnormal changes in the physiological parameter related to the physiological condition.

In an aspect, the present disclosure can include a system that can provide multimodal neuromodulation to at least one nerve of an autonomic nervous system of a patient to up regulate at least a portion of the autonomic nervous system and downregulate at least another portion of the autonomic nervous system. The system can include an electrode and a generator in communication with each other. The electrode can be in communication with the at least one nerve of the autonomic nervous system to affect a physiological condition via multimodal neuromodulation. The electrode can include at least one contact and at least one other contact. The electrode can apply a neuromodulation signal to modulate at least one physiological parameter related to the physiological condition at a time. The neuromodulation signal can have at least one signal parameter. The electrode can also apply another neuromodulation signal to modulate the at least one physiological parameter related to the physiological condition at another time. The other neuromodulation signal can have at least another signal parameter. The generator can generate and provide the neuromodulation signal, having the at least one signal parameter, to the electrode and the other neuromodulation signal, having the at least the other signal parameter, to the electrode to continuously adjust to changes in the at least one physiological parameter due to the physiological condition.

In another aspect, the present disclosure can include a method for using an electrode for bi-modal neuromodulation of at least one nerve of the autonomic nervous system that includes the following. Receiving, by a system comprising a processor, a recording of at least one physiological parameter related to a physiological condition from at least one sensor. Calculating, by the system, one or more statistical values of the at least one physiological parameter. Calculating, by the system, an output coefficient representative of a desired change in the at least one physiological parameter based on the one or more statistical values using a control system. Adjusting, by the system, at least one signal parameter of a stimulation signal and/or at least another signal parameter of a blocking signal based on the output coefficient. And, outputting, by the system, the adjusted at least one signal parameter of the stimulation signal and/or the at least the other signal parameter of the blocking signal to a generator. The generator can provide the blocking signal to the electrode at a time and the stimulation signal to the electrode at another time such that the blocking signal and the stimulation signal are applied to the at least one nerve of the autonomic nervous system to alter the at least one physiological parameter to continuously adjust to changes in the at least one physiological parameter due to the physiological condition.

In a further aspect, the present disclosure can include a system that can apply bi-modal neuromodulation of at least the vagus nerve to control a heart rate of a patient. The system can include a generator, a bi-polar electrode, at least one sensor, and a controller. The generator can generate at least a blocking signal and a stimulation signal and provide the signals to the bi-polar electrode. The bi-polar electrode can be positioned in electrical communication with the vagus nerve of the patient and can include a contact and a return contact. The contact can apply the stimulation signal to the vagus nerve to activate parasympathetic neural activity and the blocking signal to the vagus nerve to block parasympathetic neural activity. The at least one sensor can record at least an electrocardiogram (ECG) of the patient and send the recorded ECG to the controller. The controller can be in communication with the generator and the at least one sensor. The controller can include a non-transitory memory configured to store instructions and processor configured to execute the instructions. The controller can receive the recorded ECG and calculate a heart rate at a given time; calculate a median heart rate of the patient at a time based on the ECG, a median heart rate error, and a heart rate error slope; calculate an output coefficient representative of a desired change in the median heart rate error and/or the heart rate error slope using a fuzzy logic control law controller and adjust whether the stimulation signal or the blocking signal is applied and/or one or more parameters of the blocking signal and/or the stimulation signal based on the output coefficient.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram of a system for multimodal neuromodulation;

FIG. 2 is a block diagram of the system of FIG. 1 employed in a closed-loop control system;

FIG. 3 is a block diagram the system of FIG. 2 in operation;

FIG. 4 includes block diagrams of the system of FIG. 2 in use on a nerve including both afferent and efferent fibers;

FIG. 5 is a block diagram of example instructions that can be stored and executed by the controller of the system of FIG. 2;

FIG. 6 is an example use the system of FIG. 2 to upregulate and downregulate conduction in the vagus nerve;

FIGS. 7 and 8 are process flow diagrams showing methods for multi-modal neuromodulation of at least one nerve of the autonomic nervous system;

FIG. 9 is a flow diagram of an example control system that can use fuzzy logic control for closed loop control of multi-modal neuromodulation; and

FIGS. 10-16 are experimental figures demonstrating the efficacy of using a single electrode to modulate heart rate through multi-modal neuromodulation.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “neuromodulation” refers to the alteration of conduction in one or more neural structures using one or more therapy modalities (e.g., electrical, light, heat, magnetic, pharmaceutical, etc.) to cause an effect. In some instances, the altered conduction caused by the neuromodulation can be reversible. As used herein, and unless otherwise clearly stated, neuromodulation is accomplished with one or more electrical signals, each of the one or more electrical signals having at least a current or a voltage waveform configured to alter conduction of the one or more neural structures.

As used herein, the term “multimodal”, also referred to as “multi-modal”, neuromodulation refers to the alteration of conduction in a neural structure using different neuromodulation paradigms (e.g., using different electrical waveforms). For example, an electrical waveform can be configured to up-regulate a certain portion of a nerve and another electrical waveform can be configured to down-regulate another certain portion of the nerve.

As used herein, the term “block” refers to the purposeful modulation of conduction with an electrical waveform configured to decrease (or down-regulate) conduction in one or more neural structures. An example electrical waveform that is used to provide block is “kilohertz frequency alternating current block”, otherwise referred to as KHFAC block.

As used herein, the term “KHFAC block” refers to a type of block due to application of an electrical waveform configured such that upon application, one or more action potentials can be at least partially extinguished and/or otherwise prevented from propagating. The KHFAC waveform is generally a high frequency electrical waveform between 1 kHz and 100 kHz, often between 3 kHz and 60 kHz, most often between 4 Hz and 7 kHz. In some instances, KHFAC block can turn nerve conduction off and on nearly instantaneously and can modulate the effect by changing the waveform amplitude. The term blocking signal may be used synonymously with block and/or block signal.

As used herein, the term “down-regulate” refers to the process of reducing or suppressing a response to a stimulus (e.g., blocking and/or stimulating).

As used herein “stimulation” refers to the purposeful modulation of conduction to increase (or up-regulate) conduction in one or more neural structures. Electrical nerve stimulation works by providing a short electrical pulse of current and/or voltage to a nerve to trigger an action potential at the stimulation site. The stimulation of the nerve increases the frequency and number of action potential signals traveling up and/or down a collection of nerves (e.g., fibers, axons, or the like). The term stimulating signal may be used synonymously with stimulation signal.

As used herein, the term “up-regulate” refers to the process by which a neural structure increases its response to a substance or signal (e.g., blocking and/or stimulating) from outside the neural structure to carry out a specific function.

As used herein, the term “nervous system” can include the motor nervous system, which includes at least motor nerves, the sensory nervous system, which includes at least sensory nerves, and the autonomic nervous system, which includes autonomic nerves.

As used herein, the term “autonomic nervous system”, otherwise referred to as the “ANS”, refers to the network of neural structural throughout the body that control unconscious processes (e.g., breathing, heart beating, etc.) and connects the brain to most of the organs of the body. The autonomic nervous system includes the parasympathetic nervous system (PNS), the sympathetic nervous system (SNS), and, in some instances, the enteric nervous system.

As used herein, the term “sympathetic nervous system” , also referred to as “SNS”, activates body processes that help a person in times of need, especially times of stress, danger or physical activity, and is responsible for your body's “fight-or-flight” response.

As used herein, the term “parasympathetic nervous system”, also referred to as “PNS”, does the opposite of the sympathetic nervous system and activates the body process that relax the body after periods of stress or danger and runs life-sustaining processes during times when a person feels safe and relaxed. The PNS is responsible for the “rest-and-digest” body processes.

As used herein, the term “neural structure” refers to any portion of the neural systems of a patient. A neural structure can be a nerve, an axon, a fiber, a ganglionic chain, or the like that can conduct an action potential. It should be understood that while the term nerve is used throughout it may be interchangeable with any other neural structure.

As used herein, the term “nerve” refers to a bundle of fibers that send messages from parts of the body to the brain and/or vice versa in the form of electrical signals. For example, a nerve can be an autonomic nerve that can include sympathetic fibers and/or parasympathetic fibers. As another example, a nerve can be a motor nerve, a sensory nerve, or a mixed motor and sensory nerve. A nerve is an example of a neural structure.

As used herein, the term “fiber” refers to an axon, which is a long slender projection of a nerve cell or neuron in vertebrate organisms having a diameter that corresponds to conduction velocity. Generally, a fiber conducts electrical impulses transmitting information in one or more directions throughout the body and is classified depending on the type of fiber (e.g., sympathetic, parasympathetic, sensory, motor, etc.), the diameter of the fiber, and/or if myelin coating is present.

As used herein, the term “control system” refers to a set of mechanical and/or electronic devices that manage, command, direct, and/or regulate other devices or systems by way of control loops. Generally, control systems are computerized (as used herein). A control system can be open loop or closed loop. In open-loop control, the control action from the controller is independent of the “process output” (e.g., the physiological parameter). In closed loop control, the control action from the controller is dependent on the process output. Control systems can implement feedback control (e.g., Proportional-Integral-Derivative (PID) control), logic control, on-off control, linear control, and fuzzy logic control.

As used herein, the terms “fuzzy logic control”, “fuzzy logic control law”, or implemented by a “fuzzy logic control system”, otherwise referred to as FLC system, refer to one or more adaptive control systems that provide an intuitive mapping of descriptive properties of systems to the process output. Fuzzy logic is a form of many-valued logic in which the truth value of variables may be any real number between 0 and 1. Fuzzy logic is employed to handle the concept of partial truth, where the truth value may range between completely true and completely false (and any value therebetween).

As used herein, the term “physiological condition” refers to a disorder, illness, and/or injury that affects a patient causing at least one bodily function and/or response that is not considered normal. The physiological condition and/or a symptom of the physiological condition can be at least partially treated, alleviated, and/or prevented from worsening by multimodal neuromodulation. Non limiting examples of physiological conditions related to the ANS can include cardiovascular disease, hypertension, hypotension, vasomotor disorders, diabetes, hypoglycemia, and the like. Non limiting examples of physiological conditions related to the motor system can include, stroke, cerebral palsy, multiple sclerosis, spinal cord injury, and the like. Non limiting examples of physiological conditions related to the sensory system can include chronic neuropathic pain, trauma (e.g., chemical, physical, radiation, etc.) or a disease affecting one or more sensory nerve, or the like

As used herein, the term “physiological parameter” refers to a measurable characteristic of a patient that describes at least one function and/or process occurring within the patient's body. A physiological parameter of a given quantity (e.g., above or below a “normal” threshold) can be a symptom and/or cause of one or more physiological conditions. Physiological parameters related to the ANS can include, but are not limited to, heart rate, blood pressure, body temperature, glucose levels, oxygen levels, or the like. Physiological parameters related to the motor system can include, but are not limited to, force output, fatigue, gait parameters, range of movement, limb posture, or the like. Physiological parameters related to the sensory system can include, but are not limited to, sensory nerve action potentials, feelings of pain, paresthesia, or the like.

As used herein, the term “patient” can refer to any warm-blooded organism, including, but not limited to, a human being, a pig, a rat, a mouse, a dog, a cat, a goat, a sheep, a horse, a monkey, an ape, a rabbit, a cow, etc. The terms patient and subject can be used interchangeably herein.

### II. Overview

Generally, if cardiovascular diseases are left untreated or incorrectly treated, such cardiovascular diseases can severely, and even fatally, affect patients. However, cardiovascular diseases can change rapidly, making them difficult to treat. Initial treatments can include the prescription of pharmaceuticals, which are easy to take but can have dangerous side effects and/or contraindications. As the diseases progress and the patients' conditions worsen, more invasive treatment options often become necessary. Examples of these more invasive treatment options that can provide electrical stimulation directly to the heart muscles include pacemakers and implanted defibrillators. However, such options tend to be extremely invasive with stimulation leads placed directly against the heart wall and can have a number of negative side effects. There exists a need for a less invasive and safer method to effectively treat and/or control progressive cardiovascular diseases.

Instead of directly stimulating the organ itself, the stimulation can be applied to a nerve that regulates the organ. As such, stimulating the nerve can control one or more physiological conditions and/or one or more symptoms of a physiological condition. For example, neuromodulation of the upstream vagus nerve can help control heart rate, conduction speed, and contraction force in a patient with cardiovascular disease. By modulating the upstream vagus nerve, a less invasive electrode can be inserted into a patient (around and/or near a nerve as opposed to directly on and/or within the heart muscle, with roughly the same size power modules) and utilize the body's existing methods of modulating heart function. However, the vagus nerves innervate a majority of the organs of the body, not just the heart, and contain both afferent and efferent fibers. Basic stimulation of the vagus nerve indiscriminately affects conduction in both the afferent and efferent fibers, which can cause unwanted side effects. As a non-limiting example, stimulating a portion of the vagus nerve that leads to the heart to reduce tachycardie arrythmias can also activate fibers that go to the larynx, causing vocal issues. The same is true of many other nerves of the autonomic nervous system and beyond as well. Additionally, stimulation alone can only increase parasympathetic activity and cannot decrease parasympathetic activity or alter sympathetic activity.

Described herein are systems and methods that can regulate both parasympathetic signals and sympathetic signals to balance organ activity along a neural fulcrum for autonomic targets. The ANS can be particularly difficult to control with neuromodulation due to long time delays and discontinuities. For instances, the cardiovascular system is complex, and each chain of mechanisms has its own time courses and delays with effects that change over time. The alternating and/or simultaneous application of at least partial blocking and stimulation signals with an electrode as described herein can mimic the balance a body not suffering from a physiological condition would normally have while treating, alleviating, and/or preventing at least one symptom and/or portion of the physiological condition. Careful and fluid control of the at least the stimulation and blocking signals based on at least one recorded physiological parameter (which can be the symptom being treated, alleviated, or prevented) is necessary for effective multimodal neuromodulation. It should be understood that multimodal neuromodulation has applications outside the ANS and can also be used to control physiological conditions associated with the motor and/or sensory nervous systems.

### III. Systems

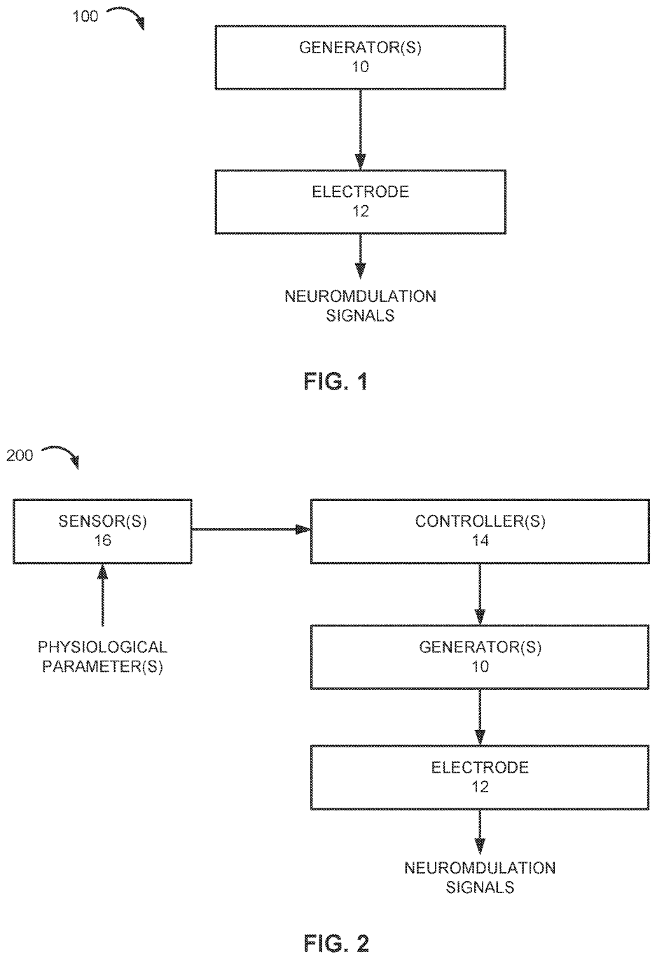

FIG. 1 shows a block diagram of a system 100 for applying multimodal neuromodulation. The system of FIG. 1 includes one or more generators (generator(s) 10) to generate two or more electrical signals that can be applied consecutively and/or simultaneously with a single electrode 12 (which can have multiple contacts). For example, the two or more electrical signals can include a signal that is configured to achieve at least partial block and another signal that achieves stimulation. The electrical signals can be designed to modulate at least one physiological parameter (e.g., heart rate, blood pressure, body temperature, glucose level, oxygen level, etc.), for example simultaneously up-and down-regulating autonomic nerve activity (e.g., vagus nerve, sympathetic chain, etc.) to reach a desired setpoint. In another example, the electrical signal can be designed to modulate at least one physiological parameter (e.g., force output, fatigue, gait parameters, range of movement, limb postures, sensory nerve action potentials, pain, paresthesia, etc.), to regulate a portion of the motor systems and/or sensory systems of the nervous system (e.g., spinal cord, peripheral motor nerves, peripheral sensory nerves, peripheral mixed motor and sensory nerves, etc.). It will be understood that the terms “electrical signal” and “neuromodulation signal” can refer to the same type of electrical signal that can be generated with at least one altered parameter and applied to a nerve to cause neuromodulation.

The single electrode 12 can provide the multiple electrical signals (e.g., neuromodulation signals) simultaneously for neuromodulation of at least one nerve of the autonomic nervous system, the motor system, and/or the sensory system. In some instances, modulation of parameters of the multiple electrical signals (e.g., frequency modulation of the stimulation waveform and amplitude modulation of the blocking waveform) can successfully titrate the effects on the at least one physiological parameter. Observed physiological effects (e.g., of the at least one physiological parameter) can be translated to a control scheme for effective treatment, alleviation, and/or prevention of at least a portion of a physiological condition causing and/or caused by an abnormal physiological parameter. Consequently, the combination of upregulation and downregulation of the nervous system can provide completely control to drive the nervous system to dynamic set points and/or maintain the system at homeostasis in response to external perturbation (e.g., a physiological condition).

The system 100 can include the at least one generator (e.g., generator(s)) 10 in communication with the electrode 12 to apply two or more electrical signals to at least one nerve (not shown) to affect a physiological condition. The at least one generator 10 can be in electrical communication (wired and/or wireless) with the electrode 12 and can provide the two or more electrical signals to the electrode. It should be understood that each electrical signal can be generated and provided by a single generator and/or a single generator can generate and provide more than one electrical signal to the electrode depending on the type(s) of generators. The at least one generator 10 can have one or more signal channels (not shown), and each of the one or more signal channels can be connected to the electrode 12 such that one channel may carry a single electrical signal and/or more than one electrical signal. The electrode 12 can include at least two contacts in electrical communication with the at least one nerve. As an example, one of the at least two contacts can be the signal applying contact and another of the at least two contacts can be a return contact to localize the effects of the applied electrical signal. Optionally, the at least two contacts of the electrode 12 can separately receive electrical signals from the at least one generator 10 and a third contact and/or an additional electrode can act as the return.

For instance, the at least one generator 10 can generate and provide an electrical signal (e.g., a neuromodulation signal) including at least one variable signal parameter to the electrode 12 and generate and provide another electrical signal (e.g., another neuromodulation signal) including at least another variable signal parameter to the electrode. The electrical signal and the other electrical signal can be provided at different times and/or simultaneously. The electrode 12 can apply the electrical signal including the at least one variable signal parameter to the nerve to modulation at least one physiological parameter related to the physiological condition at a time. The 12 can apply the other electrical signal to modulate the at least one physiological parameter related to the physiological condition at another time, at the same time, or an overlapping time. For example, the electrical signal can up regulate the at least one physiological parameter and the other electrical signal can downregulate the at least one physiological parameter and/or the another at least one physiological parameter. Additionally and/or alternatively, the electrical signal can activate a parasympathetic response and the other electrical signal can activate a sympathetic response or attenuate the parasympathetic response, or vice versa. In some instances, the electrical signal can be a stimulation signal and the other electrical signal can be a blocking signal. In other instances, the electrical signal can be a blocking signal and the other electrical signal can be a stimulation signal.

The electrode 12 can be any multi-contact electrode that can be in electrical communication with a desired nerve for treating, alleviating, and/or preventing an abnormal value in at least one physiological parameter related to the physiological condition. For example, the electrode 12 can be a nerve cuff electrode, a disc electrode, a loop electrode, a needle electrode, or the like. The electrode 12 can be a biocompatible, implanted electrode. For example, the at least two contacts of the electrode 12 can be in direct contact with (e.g., touching), direct communication (e.g., space between the contact and the nerve, but no intervening tissue), and/or adjacent (e.g., may include some intervening tissue between the contact and the nerve. While not wishing to be bound by theory, implanting an electrode 12 around, in contact with, and/or near a nerve are generally significantly less invasive procedures than procedures that may directly ablate a portion of an organ to remove the effect from one or more nerves or require implantation of a device directly to an organ (e.g., a pacemaker or the like).

The nerve (generally referred to as a nerve singular for ease of illustration and understanding but may be more than one nerve and/or another type of neural structure) can be a nerve of the autonomic nervous system. For example, the nerve can be a vagus nerve, a portion of the sympathetic chain ganglia, or the like. The nerve can be able to affect at least one physiological parameter such as, heart rate, blood pressure, body temperature, glucose level, or the like. In the case of a patient living with a physiological condition the at least one physiological parameter can be a symptom and/or a cause of the physiological condition and is considered abnormal if not treated, alleviated, and/or prevented through some means. For example, the physiological condition can be, hypertension, hypotension, bradycardia, tachycardia, arrythmia, diabetes, hypoglycemia, a vasomotor condition, or the like. For instance, the physiological condition can be a cardiovascular disease (e.g., including at least one of bradycardia, tachycardia, arrythmia, or the like) and the physiological parameter can be heart rate.

In another example, the nerve can be a nerve of the motor system (e.g., peripheral nerve, spinal cord, or the like) related to a limb effected by a physiological condition such as stroke, cerebral palsy, multiple sclerosis, spinal cord injury or the like that can cause muscle spasticity and at least partial motor function degeneration and/or loss. At least one symptomatic physiological parameter can include muscle force, gait parameters, fatigue, range of movement, limb posture, pain, or the like. Bi-modal neuromodulation can at least partially relieve spasticity and restore motor function.

In a further example, the nerve can be a nerve of the sensory system (e.g., peripheral nerve, spinal cord, or the like). The physiological condition can be pain related to chronic neuropathy, trauma (e.g., physical, chemical, radiation, post-surgical, or the like), disease, or the like. Bi-modal neuromodulation can at least partially treat and/or alleviate pain while not impacting function or impacting function less than current methods. At least one physiological parameter that can be monitored can include sensory nerve action potentials, feelings of pain and/or paresthesia, or the like.

FIG. 2 shows a system 200 that can include the generator(s) 10 and the electrode 12 of system 100. System 200 can further include at least one controller (e.g., controller(s)) 14 and at least one sensor (e.g., sensor(s)) 16. The at least one sensor 16 and at least one controller 14 can create closed loop control. However, it will be understood that the at least one controller 14 may be an open loop system that requires a user input before proceeding.

The at least one controller 14 can be in electrical communication (wired and/or wireless) with the at least one generator 10 and the at least one sensor 16. The at least one sensor 16 can record the at least one physiological parameter and can send the recording of the at least one physiological parameter to the controller. The at least one sensor 16 can measure at least one of heart rate, blood pressure, glucose levels, muscle force, sensory nerve signals, or the like. The at least one sensor 16 can include at least one sensing electrode, at least one position sensor, at least one motion sensor, or the like. For example, the at least one sensor 16 can record an electrocardiogram (ECG) of a patient. In some instances, the at least one sensor 16 can continuously record the at least one physiological parameter and send the recording to the at least one controller (e.g., at frequency in given time increments on the order of milliseconds, seconds, minutes, etc.). In other instances, the at least one sensor 16 can record the at least one physiological parameter and/or send the recording when queried by the controller or another device (e.g., manually through some other connected device (not shown) by the patient, a caregiver, a medical professional, or the like) to check on the at least one physiological parameter. The at least one sensor 16 can be positioned on the skin, percutaneous, and/or implanted. The at least one sensor 16 can include a battery, a wireless transceiver, and/or any other circuitry needed for implementation. When the at least one sensor 16 is more than one sensor, each of the sensors can be positioned at a same location and/or different locations, depending on the physiological parameters to be recorded, and can record a single physiological parameter with multiple sensors and/or each record a different physiological parameter.

The at least one controller 14 can include at least a non-transitory memory (not shown in FIG. 2) for storing instructions related to the multi-modal neuromodulation and a processor (not shown in FIG. 2) for executing the instructions. Each of the at least one controller 14 can include at least the circuitry and/or power components (e.g., battery and/or plug into the wall) to run the controller and/or the system 200 as a whole. The at least one controller 14 can also include at least one of a user interface (e.g., touch screen, buttons, keyboard, mouse, microphone, etc.), a visual display, a speaker, a haptic feedback device, a wireless transceiver, a wearable connector (e.g., arm band, wrist band, leg band, belt clip, or the like), or the like. The at least one controller 14 can receive the recorded at least one physiological signal from the at least one sensor 16, which may indicate when and adjustment of the neuromodulation signals applied through the electrode 12 is necessary. The at least one controller 14 can adjust the at least one signal parameter of the neuromodulation signal and/or the at least the other signal parameter of the other neuromodulation signal based on the at least one physiological parameter (discussed in more detail below). The at least one controller 14 can be one controller connected to one generator (of generator(s) 10), one controller connected to multiple generators, multiple controllers with each controller connected to each of the multiple generators, or the like.

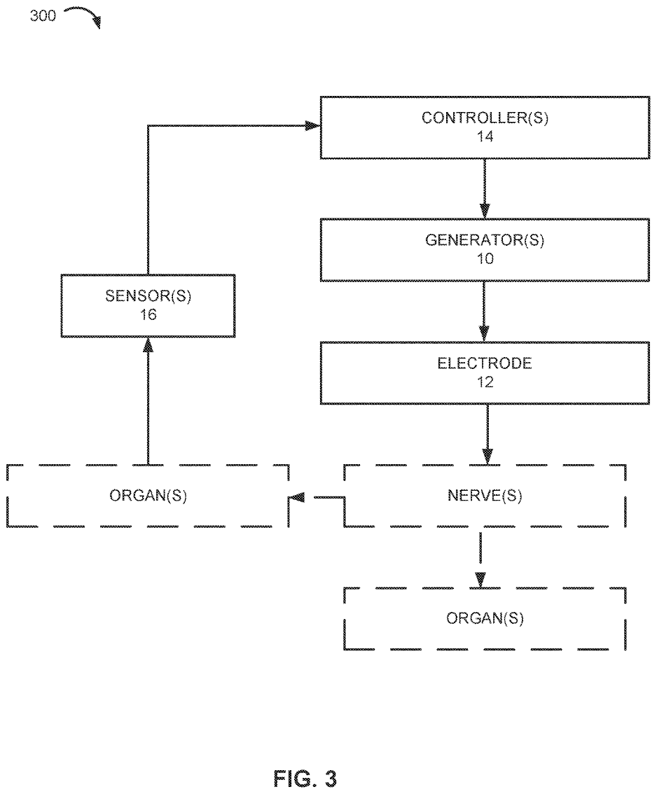

FIG. 3 shows a block diagram of the system 300 in use. The system 300 can be basically the same as system 200. The electrode 12 can apply at least the electrical signal (e.g., the neuromodulation signal) and the other electrical signal (e.g., the other neuromodulation signal) to at least one nerve. The at least one nerve can communicate signals to one or more organs via the same or different fibers within the nerve. In some instances, the electrical signal and the other electrical signal can affect conduction of signals to and/or from one organ. In other instances, the electrical signal and the other electrical signal can affect conduction of signals to and/or from a plurality of organs, which may be the same and/or different, depending on the types of neuromodulation signals and the nerve(s) in question. Basic stimulation and/or block of many nerves indiscriminately affects more fibers than needed (e.g., efferent fibers to additional organs outside the target organ, afferent fibers to the brain not meant to be affected, or the like) and causes side effects, which may be a significant deterrent to the use of neuromodulation as a treatment. The use of multiple simultaneous neuromodulation signals can selectively activate and/or block of conduction through one or more fibers to affect only desired organs and physiological parameters and/or can balance the activity of the parasympathetic nervous system and the sympathetic nervous system.

For example, the vagus nerve (cranial nerve X) is one of the main neural inputs into the heart that control cardiac function and can be a target for neuromodulatory treatment in cardiac disease. There is a vagus nerve on each side of the body, both originating from the medulla of the brainstem, and traveling down through the neck and into the whole body. The vagus nerve (like all nerves) is composed of many nerve fibers (or nerve cells) that each travel the whole length from the brain to innervation targets. The fibers in the vagus nerves innervate a majority of the organs in the body including most notably the heart, the lungs, and the organs of the digestive tract. The vagus nerve also branches into the pharynx, the larynx, the spine, and the ear. As such the vagus nerve is a very important part of sending signals to and from the brain to the body and stimulation of the vagus nerve can indiscriminately stimulate both afferent and efferent fibers and cause affects in multiple organs (e.g., side effects). In some instances, basic stimulation intended to activate efferent fibers carrying information to heart can also activate afferent fibers carrying information to the brain or efferent fibers carrying information to another organ, such as the larynx, causing hoarseness. Additionally, stimulation of the vagus nerve increases parasympathetic activity while block can decrease parasympathetic activity. In order to implement complete control, both are needed.

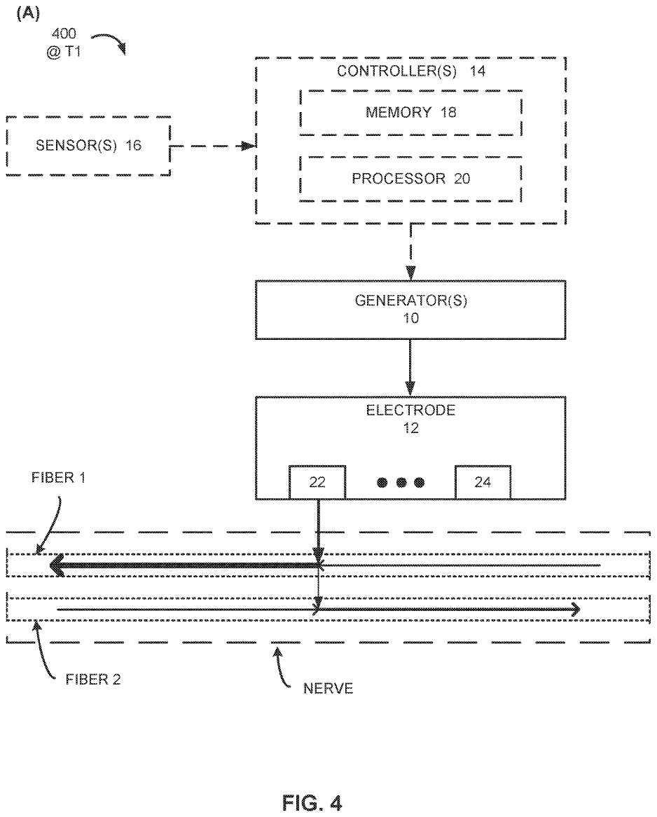

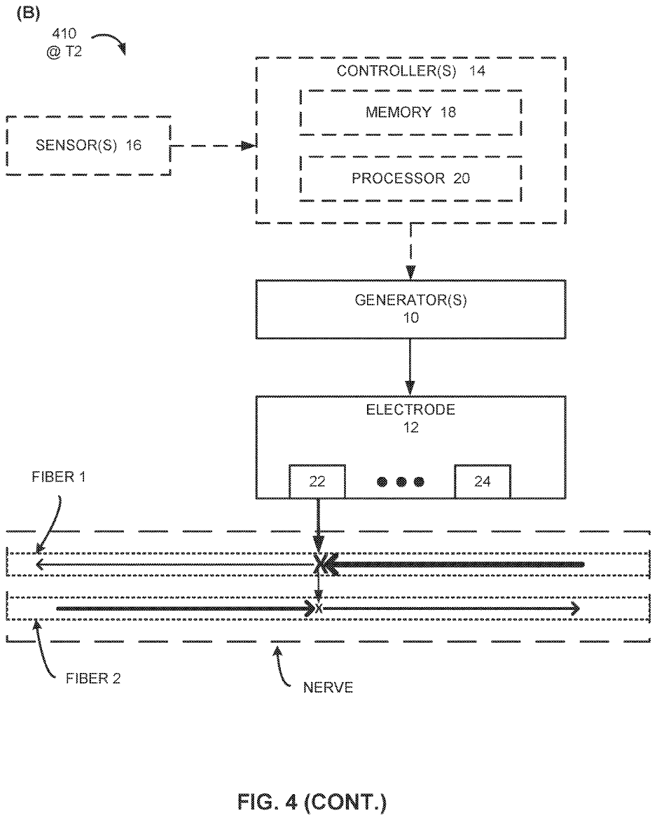

FIG. 4, elements A and B, show the application of two electrical signals for neuromodulation of a nerve with system 400 and an example of effects of the two neuromodulation signals in more detail. FIG. 4, element A shows an example of stimulation at a time (T1) and FIG. 4, element B shows an example of block at another time (T2), the times can be in any order, overlapping, and/or simultaneous. The system 400 can be the system 100 or the system 200. For ease of illustration, two fibers are shown, each having conduction in a different direction but it should be understood that a nerve can have any number of fibers in either direction and that stimulation and/or block can affect one or more fibers in either direction depending on the signals used. The electrode 12 can include at least two contacts, at least one contact 22 and at least one other contact 24, in electrical communication with a nerve. While only two contacts 22 and 24 are shown in FIG. 4, with contact 22 being the signal applying contact and contact 24 being the return contact, it should be understood the electrode 12 can include any number of contacts and more than one contact can be a signal applying contact. As shown in FIG. 4, element A, the at least one contact 22 can apply a neuromodulation signal to a first fiber (Fiber 1), which can be an electrical stimulation signal in this example, such as a stimulation current. The stimulation signal can have signal parameters including, but not limited to frequency, amplitude, pulse duration, waveform shape, and length of application. As an example, the frequency can be 100 Hz or lower, 80 Hz or lower, 60 Hz or lower, or the like; the amplitude can be between 0 mA and 20 mA, between 0 mA and 10 mA, or the like; and the pulse duration can be between 0 μs and 10 μs, 0 μs and 100 μs, 0 μs and 200 μs, 0 μs and 500 μs, or the like.

As shown in FIG. 4, element B, the at least one contact 22 can apply another neuromodulation signal to the second fiber (Fiber 2), which can be a blocking signal in this example, such as a kilohertz frequency alternating current (KHFAC) block. The blocking signal can have signal parameters including, but not limited to, frequency, amplitude, pulse duration, waveform shape, and length of application. The effect of KHFAC block can vary depending on the waveform shape and frequency. Lower amplitudes can be used for block with lower frequency (~10 kHz) and with square waveforms due to the larger charge per cycle. Higher frequency blocking waveforms require more current, but often have less onset when tested in the motor system. As an example, a KHFAC block can have a frequency between 1 kHz and 100 kHz, between 3 kHz and 60 kHz, between 4 Hz and 7 kHz, between 5 kHz and 100 kHz, between 5 kHz and 50 kHz, between 10 kHz and 60 kHz, or the like; and an amplitude between 0 mA and 20 mA, between 0 mA and 10 mA, between 10 mA and 20 mA, or the like. For instance, the KHFAC block can have a frequency of 5 kHz or higher and an amplitude between 0 mA and 20 mA.

While not wishing to be bound by theory it is noted that In general, amplitude modulation of a waveform effects how many individual nerve fibers in a nerve tract are activated, as some fibers require more current than others to activate (small fibers and fibers deeper within the nerve tract require more current). Frequency modulation at the high amplitudes discussed herein activate all fibers but varies how often the fibers are activated. Each approach has its advantages and disadvantages, but either can be used depending on the specific nerve and physiological parameter(s) to be controlled.

The stimulation signal can at least partially increase conduction in at least one fiber within the nerve (e.g., Fiber 1). The conduction can be increased anywhere between 0 and 100% depending on the situation. The blocking signal can at least partially decrease and/or cut off conduction in at least one other fiber within the nerve (e.g., Fiber 2). The conduction can be decreased anywhere between 0 and 100% depending on the situation. The stimulation signal can, for example, activate a parasympathetic response and the blocking signal can attenuate a parasympathetic response in the autonomic nervous system. Application of both stimulation and block, used consecutively, overlapping in time, and/or simultaneously can balance parasympathetic and sympathetic responses in a “normal” manner. For instance, stimulation of the vagus nerve alone can only increase parasympathetic responses and block of the vagus nerve along can only decrease parasympathetic responses, which is ineffective for treatment when a physiological condition can cause the at least one physiological parameter to swing between abnormalities in either augmented or attenuated responses. The levels of parasympathetic and sympathetic activation can be chosen and/or adjusted based on a manual input (e.g., by a patient, caregiver, medical professional, or the like) and/or in a feedback loop based on the at least one physiological parameter recorded by the at least one sensor(s) 16 at any given time. The stimulation or blocking signal to be applied at the given time and/or the parameters of the stimulation and/or blocking signal can be chosen and/or adjusted based on the desired parasympathetic and/or sympathetic responses to treat, alleviate, and/or prevent at least a portion of a physiological condition. It should be understood also that one or more stimulation signals and/or blocking signals can be used with one or more different parameters for different effects on the nerve and/or fibers of the nerve.

Using the vagus nerve as an example, stimulation of the vagus nerve can reduce heart rate by increasing parasympathetic activity and activating efferent fibers that innervate the heart. However, as mentioned above, basic stimulation with cuff electrode activates efferent fibers indiscriminately with afferent vagal fibers (fibers that carry information from the body to the brain) as well as efferent fibers that go to organs other than the heart. These afferent fibers can cause unwanted side effects, such as hoarseness of the voice from activating fibers that go to the larynx in the throat. At least partially blocking the afferent fibers and/or the not intended to be innervated efferent fibers can remove unwanted side effects from vagus nerve stimulation and provide a more natural balance of sympathetic and parasympathetic neural activity. Moreover, stimulation can only increase parasympathetic activity while block is needed to decrease parasympathetic activity.

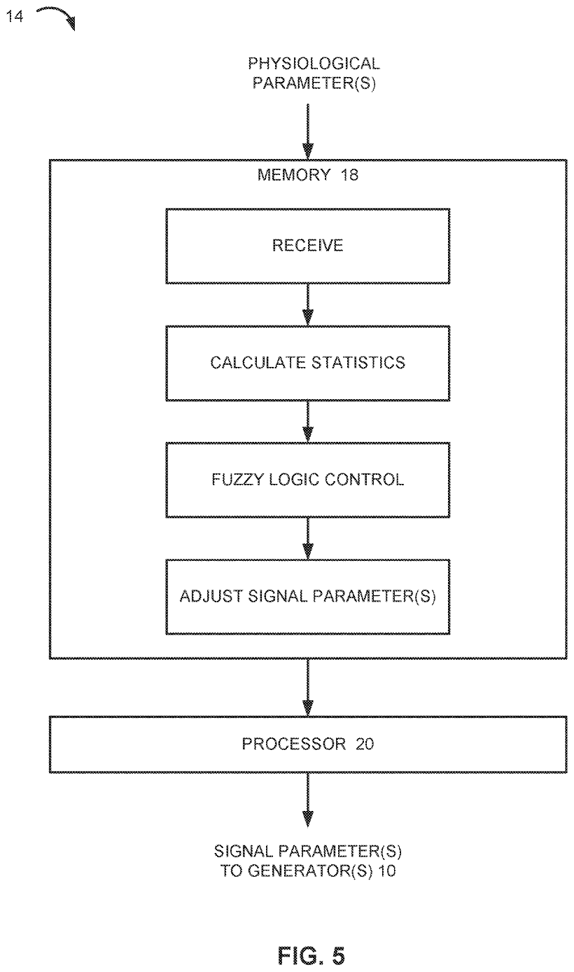

The controller(s) 14 can adjust the signal parameters based on feedback from the patient. FIG. 5 shows an example of one of the controller(s) 14 in further detail. It should be understood that each of the at least one controller(s) 14 previously discussed can have the same components and/or instructions as described here. The controller 14 can include a non-transitory memory 18 for storing instructions and a processor 20 for executing the instructions. As previously mentioned, the controller can also include other components not shown in FIG. 5 including, but not limited to, a user interface, a display, a wireless transceiver, power component(s), or the like. The controller 14 can be used to set initial signal parameters for each of the at least two neuromodulation signals (e.g., chosen by a medical profession for the patient), choose which of the at least two neuromodulation signals is applied at a given time, and adjust at least one of the signal parameters of at least one of the at least two electrical signals (e.g., neuromodulation signals) over the duration of the use of the system (e.g., system 200).

To choose which of the at least two neuromodulation signals is applied at a given time and/or adjust the at least one signal parameter the instructions stored in the memory 18 can include receiving a recording of the at least one physiological parameter (e.g., related to the physiological condition) from the at least one sensor (e.g., sensor(s) 16, not shown in FIG. 5). The controller 14 (and/or the sensor itself) can include instructions for a frequency of receiving the at least one physiological parameter. In some instances, the controller 14 can include an instruction to query the at least one sensor for the physiological parameter in response to a set condition being met and/or a manual input (e.g., through a user interface). The controller 14 can store a plurality of recordings of the at least one physiological parameter in a buffer (e.g., the most recent 1 second of recordings, the most recent 2 seconds of recordings, the most recent 5 seconds of recordings, the most recent 10 seconds of recordings, etc.). The controller 14 can then calculate at least one statistical value for that at least one physiological parameter at a given time based on the recordings of the at least one physiological parameter. For example, the median and/or the error of the at least one physiological parameter at the time (e.g., over the most recent set of recordings) can be calculated using the most recent recordings stored in the buffer. In some instances, if the at least one physiological parameter needs to be calculated from a recording (e.g., heart rate from an ECG recording) then the controller can do this prior to calculating the at least one statistical value. If an error in the recording or the calculation is determined (e.g., outside a normal threshold range), then the instruction can use the last known good calculation and/or recording. From the median (or other statistical value) a smoothed average of the at least one physiological parameter can be calculated that is unaffected by momentary changes. A physiological parameter error can be further calculated based on the difference between a set point for the physiological parameter and the median of the physiological parameter calculated for that time. And a slope of the error of the physiological parameter can be calculated compared to the most recent previous median error of the physiological parameter.

The controller 14 can then calculate an output coefficient representative of a desired change in the at least one physiological parameter using a control system based at least in part on the at least one statistical value of the at least one physiological parameter (e.g., the median, the error, the slope, or the like). The control system can use, for instance, fuzzy logic control. The fuzzy logic control can include four zones with each zone responding to a combination of high/low physiological parameter (e.g. compared to normal) and increasing/decreasing physiological parameter (e.g., compared to number of the most recent previous recording(s)) (e.g., (1) high physiological parameter that is increasing, (2) high physiological parameter that is decreasing, (3) low physiological parameter that is increasing, and (4) low physiological parameter that is decreasing. The output coefficient can be determined based on where the calculations fall within the four zones and can be any number between −1 and 1. Additionally, when the error of the physiological parameter relates to an output coefficient that is considered “good” (e.g., within a predetermined range around 0 considered to be good for the patient and/or the physiological condition) then the control system can dampen and/or not make adjustments. It should be noted that a four-zone fuzzy logic control system is only one example and other types of control logic and/or number of zones in a fuzzy logic control system can be used depending on the complexity, number of statistical values used in the calculation and/or number of physiological parameters evaluated.

The controller 14 can determine if the electrical signal being applied needs to be changed (e.g., from stimulation to block or vice versa) and/or adjust the at least one signal parameter of the electrical signal and/or the at least the other signal parameter of the other electrical signal based on the output coefficient. For instance, when the output coefficient is positive, the neuromodulation signal (e.g. the stimulation signal) can be applied and/or at least one signal parameter of the neuromodulation signal can be increased; when the output coefficient is zero, no action is taken; and when the output coefficient is negative, the other neuromodulation signal (e.g., the blocking signal) can be applied and/or at least the other signal parameter of the other neuromodulation signal can be increased. When the output is positive and above a given threshold (e.g., close to 1) then, the at least one signal parameter of the neuromodulation signal (e.g., the stimulation signal) can be increased (e.g., because the neuromodulation signal was already being applied but was not having enough of an effect). Optionally, the at least the other signal parameter of the other neuromodulation signal can be decreased also if both signals are applied at overlapping and/or simultaneous times. When the output coefficient is negative and below another threshold (e.g., close to −1), then the at least the other signal parameter of the other neuromodulation signal (e.g., the blocking signal) can be increased (e.g., because the other neuromodulation signal was already being applied, but was not having enough of an effect). Optionally, the at least one signal parameter of the neuromodulation signal can be decreased.

In another instance, the signal parameters of the electrical signal and/or the other electrical signal can be increased or decreased as necessary to make the effects of the signals be increased or decreased. For example, when the output coefficient is positive, the neuromodulation signal can be applied and/or the effect of the neuromodulation signal can be increased; when the output coefficient is zero, no action is taken; and when the output coefficient is negative, the other neuromodulation signal can be applied and/or the effect the other neuromodulation signal can be increased. When the output is positive and above a given threshold (e.g., close to 1) then, the neuromodulation signal can be increased more and/or the other neuromodulation signal can be decreased also. When the output coefficient is negative and below another threshold (e.g., close to −1), then the other neuromodulation signal can be increased more and/or the neuromodulation signal can be decreased also. For instance, the frequency of a stimulation waveform can be increased or decreased to increase or decrease an effect of a stimulation signal, respectively. An amplitude of a block waveform can be increased or decreased to increase or decrease the blocking effect, respectively. The adjusted signal parameter(s) can then be sent to the generator(s) to be applied through the contacts of the electrode.

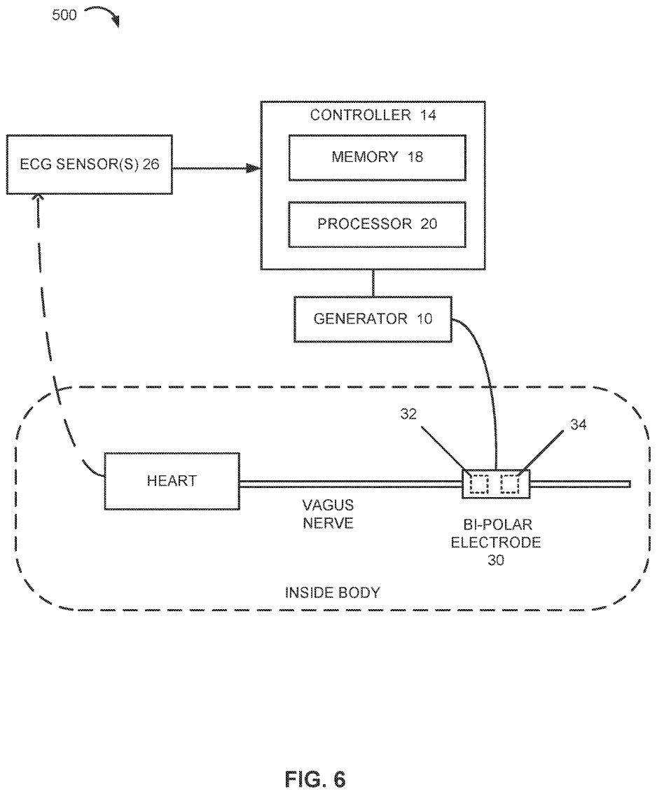

FIG. 6 shows an example system 500 for applying bi-modal neuromodulation to the vagus nerve to control a heart rate of a patient having a cardiovascular disease. The system 500 can be a closed loop feedback system that can check the heart rate and adjust the bi-modal neuromodulation based on the sensed heart rate. The system 500 can include a generator 10 that can generate at least a blocking signal and a stimulation signal. The generator 10 can be in electrical communication (wired and/or wireless) with a controller 14 and a bi-polar electrode 30. The controller 14 can include a non-transitory memory 18 for storing instructions and a processor 20 for executing the instructions (as described above with respect to FIG. 5), and any of the components previously mentioned. The bi-polar electrode 30 can be positioned in electrical communication with a vagus nerve of a patient. For example, as shown the bi-polar electrode can be a nerve cuff wrapped around a portion of the vagus nerve at a point that can innervate the heart. The bi-polar electrode 30 can include a contact 32 and a return contact 34 that can be near (with or without any intervening tissue between) and/or directly contacting the vagus nerve. The location of the contact 32 and the return contact 34 can be switched in some instances. The contact 32 can apply the stimulation signal to the vagus nerve to activate parasympathetic neural activity and can apply the blocking signal to the vagus nerve to attenuate parasympathetic neural activity.

The ECG sensor(s) 26 can be at least one sensor that can record an electrocardiogram (ECG) of the patient from any location in and/or on the patient. The electrocardiogram of the patient can be recorded over time at a chosen frequency. The controller 14 can receive the recorded ECG from the ECG sensor(s) and then calculate a median heart rate of the patient at a time based on the ECG and a heart rate error; calculate an output coefficient representative of a desired change in the median heart rate and/or the heart rate error using a fuzzy logic control system to adjust one or more parameters of the blocking signal and/or the stimulation signal based on the output coefficient. To calculate the median heart rate of the patient at a time the controller 14 can sample the ECG at a frequency for in time segments (e.g., 1 kHz in 0.5 second segments). Each of the segments can be loaded into a buffer such that the most recent time period of segments (e.g., 1 second, 2 seconds, 3 seconds, 5 seconds, or the like) are retained and the oldest segment(s) are removed. The segments of the ECG in the buffer can be used for the calculation of the heart rate by taking the mean of the inter-pulse times between successive QRS complex peaks. If the heart rate is outside of an expected value (e.g., outside 75 bpm-400 bpm), then the controller can deem an error occurred (e.g., due to electrical interference on the ECG signal) and the last known good heart rate calculation can be used instead. The median of the last predetermined number of heart rate calculations (e.g., the last four heart rate calculations determined from the last 4 segments saved within the 2 second buffer, or the like) can be used as input for further calculations. By taking the median heart rate over the time period a smoothed average heart rate can be obtained that can be unaffected, or less affected, by momentary changes in heart rate (e.g., premature ventricular contractions, sinus arrythmia from breathing, or the like). A heart rate error can be calculated based on the difference between a predetermined heart rate setpoint and the median heart rate. A heart rate slope (or rate of change of the heart rate error) can be calculated based on the most recent median heart rate minus the previous median heart rate that was calculated.

The heart rate slope and the heart rate error can be input into the fuzzy logic control system to output an output coefficient that represents a desired change in the heart rate. The output coefficient can be between −1 and 1. The positive values can correspond with a stimulating signal (e.g., a low frequency, activating stimulation) and the negative values can correspond with a blocking signal (e.g., a KHFAC block waveform). The larger the absolute value, the stronger the effect desired from the signals becomes (e.g., an increasing positive value will increase the frequency of the activating stimulation and a decreasing negative value will increase the amplitude of the blocking waveform). In some instances, the output coefficients can be normalized at a refresh rate and an additional gain can be applied such that at full power a maximum change in heart rate would be a predetermined bpm over a predetermined time period (e.g., 5 bpm over a 1 second period).

The fuzzy logic control system can include, for example, four “zones”, that each respond to a combination of high/low heart rate error and increasing/decreasing heart rate slope (e.g. (1) high heart rate error that is increasing, (2) high heart rate error that is decreasing, (3) low heart rate error that is increasing, and (4) low heart rate error that is decreasing). When the heart rate is deemed to be “good” (e.g., within a predetermined range around 0 considered to be good for the patient and/or the physiological condition) then the control system can dampen any adjustments and/or not make adjustments to the stimulating and/or blocking signal parameters. For example, with these damping effects, any heart rate with 5 bpm from a “good” setpoint would elicit little or no change to the signal parameters of the stimulating and/or blocking signals, but changes would increase if the heart rate error increased. It should be noted that the fuzzy logic control system can produce output coefficients all along a gradient of high/low heart rate error and high/low heart rate slope and only some example general guidelines are described below. It should be noted that any increases and/or decreases in signal parameters can be related to how close the output coefficient is to −1, 0, or 1.

If the output coefficient is a large positive value (e.g., within a first predetermined value and 1, the zone (1) described above) then the feedback indicates the heart rate error is high and the slope of the heart rate error is high, and the heart rate is high and trending higher and needs to be significantly lowered. Then the controller 14 can output signal parameters to increase the frequency of the stimulating waveform to increase the parasympathetic response, and optionally, if applied simultaneously, decrease the amount of block (e.g., decrease the amplitude of the block waveform) to increase the parasympathetic response. When the output coefficient is positive but between the first predetermined value and about 0 (e.g., the thresholds around 0 considered “normal” for the patient”) (e.g., in the zone (2) described above) then the feedback indicates the heart rate error is high, but the slope of the heart rate error is decreasing. The controller 14 can output signal parameters that can apply a stimulation signal if not already applied, increase the frequency of the stimulating waveform (but less so than in zone 1) to increase the parasympathetic response, and/or lower the amount of block (e.g., lower the amplitude of the block waveform) to slightly increase the parasympathetic response.

When the output coefficient is a negative value between about 0 (e.g., the thresholds around 0 considered “normal” for the patient”) and a second predetermined threshold (e.g., in zone (3)) indicating that the heart rate is low but increasing. Then, the control 14 can output signal parameters that can apply a blocking signal if not already applied, increase the blocking signal (e.g., increase the amplitude of the block waveform, but less so than for zone (4) described below) to increase the sympathetic response and/or attenuate the parasympathetic response, and/or decrease the frequency of the stimulating waveform to slightly decrease the parasympathetic response. When the output coefficient is near negative 1 (e.g., between negative 1 and the second predetermined threshold) indicating the heart rate is low and trending lower (e.g., in zone 4). Then, the controller 14 can output signal parameters that can increase the blocking signal (e.g., increase the amplitude of the block waveform) to increase the sympathetic response and/or attenuate the parasympathetic response, and optionally, if applied simultaneously, decrease the frequency of the stimulating waveform to decrease the parasympathetic response. These adjustments to the heart rate can continuously update to provide the patient with as normal a heart rate as possible (e.g., within predetermined thresholds). It should be noted that controller may be in communication with an external device (e.g., containing a screen, light, speaker, haptic feedback device, or the like) and/or contain an alert device (e.g., audio, visual, and/or haptic) that can be triggered if the bi-modal neuromodulation does not bring the heart rate within acceptable safety bounds within a predetermined time. The alerts can go to the patient, a caregiver, a medical professional, emergency personnel or the like.

### IV. Methods

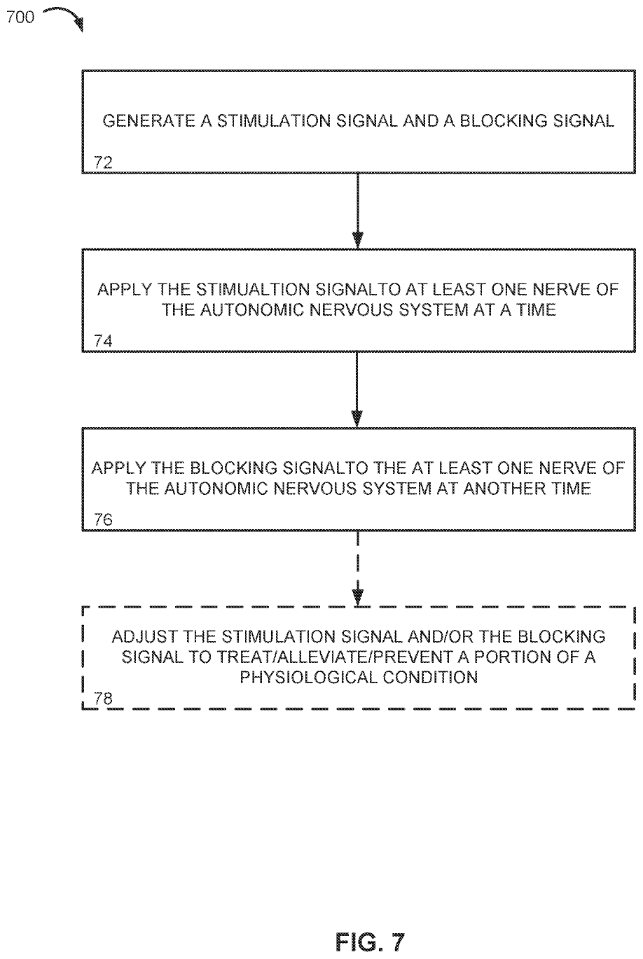

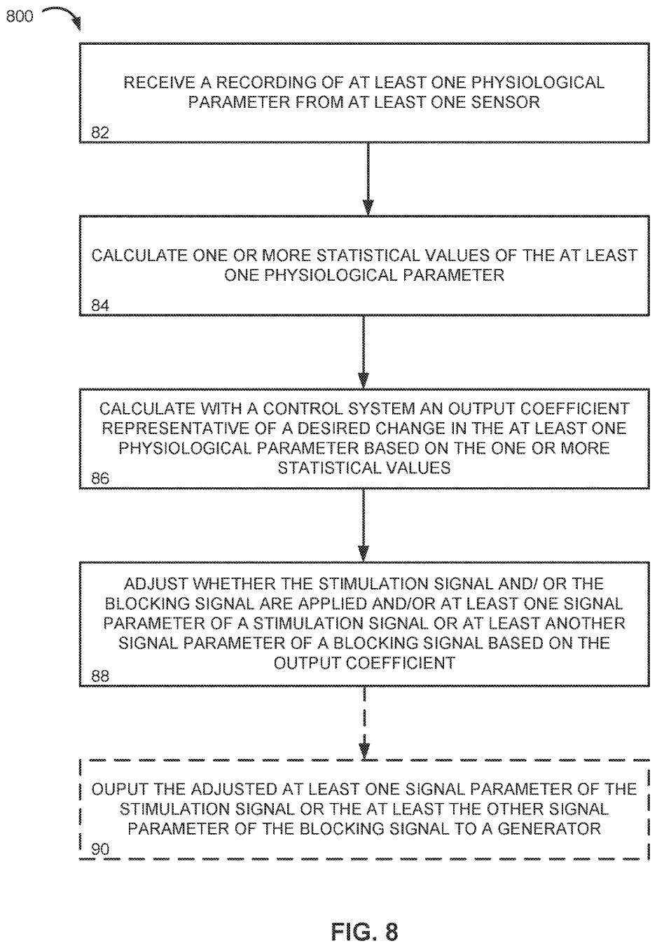

Another aspect of the present disclosure can include methods 700 and 800 (FIGS. 7 and 8) for using a multi-contact electrode to provide multi-modal neuromodulation to a nerve of the autonomic nervous system (and/or motor nervous system and/or sensory nervous system), and method 900 for fuzzy logic control of multi-modal neuromodulation. Bi-polar neuromodulation with two signals is described herein, but it should be understood that any number and/or combination of neuromodulation signals can be used for these methods. The methods can use the systems previously described (e.g., system 100 or system 200) for execution. The methods can treat, prevent, and/or alleviate a symptom and/or a portion of a physiological condition (e.g., related to an abnormal physiological parameter).

The nerve can be, for example the vagus nerve or the sympathetic ganglionic chain. The nerve can be able to affect at least one physiological parameter such as, heart rate, blood pressure, body temperature, glucose level, or the like. In the case of a patient living with a physiological condition the at least one physiological parameter can be a symptom and/or a cause of the physiological condition and is considered abnormal if not treated, alleviated, and/or prevented through some means. For example, the physiological condition can be, hypertension, hypotension, bradycardia, tachycardia, arrythmia, diabetes, hypoglycemia, a vasomotor condition, or the like. For instance, the physiological condition can be a cardiovascular disease (e.g., including at least one of bradycardia, tachycardia, arrythmia, or the like) and the physiological parameter can be heart rate.

In another example, the nerve can be a nerve of the motor system (e.g., peripheral nerve, spinal cord, or the like) related to a limb effected by a physiological condition such as stroke, cerebral palsy, multiple sclerosis, spinal cord injury or the like that can cause muscle spasticity and at least partial motor function degeneration and/or loss. At least one symptomatic physiological parameter can include muscle force, gait parameters, fatigue, range of movement, limb posture, pain, or the like. Bi-modal neuromodulation can at least partially relieve spasticity and restore motor function. In a further example, the nerve can be a nerve of the sensory system (e.g., peripheral nerve, spinal cord, or the like). The physiological condition can be pain related to chronic neuropathy, trauma (e.g., physical, chemical, radiation, post-surgical, or the like), disease, or the like. Bi-modal neuromodulation can at least partially treat and/or alleviate pain while not impacting function or impacting function less than current methods. At least one physiological parameter that can be monitored can include sensory nerve action potentials, feelings of pain and/or paresthesia, or the like.

For purposes of simplicity, the methods are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method, nor is the method necessarily limited to the illustrated aspects.

Referring now to FIG. 7, illustrated is a method 700 for providing multi-modal neuromodulation in the form of bi-polar neuromodulation to a nerve of the autonomic nervous system using at least one generator (e.g., generator(s) 10) and a bi-polar electrode (e.g., electrode 12 or 30). At 72, a stimulation signal having at least one signal parameter and the blocking signal have the at least another signal parameter can be generated. The stimulation signal and the blocking signal can be generated by a single generator and/or different generators. The at least one signal parameter of the stimulation signal can include, but is not limited to, at least one of frequency, amplitude, pulse duration, waveform shape, and time of application. The at least the other signal parameter of the blocking signal can include, but is not limited to, at least one of frequency, amplitude, pulse duration, waveform shape, and time of application. At 74, the stimulation signal having the at least one signal parameter can be applied to the at least one nerve of the autonomic nervous system by the electrode at a time. The stimulation signal can be a stimulation current waveform. At 76, the blocking signal having the at least the other signal parameter can be applied to the at least one nerve of the autonomic nervous system by the electrode at another time. The blocking signal can be a kilohertz frequency alternative current (KHFAC) block. The stimulation signal and the blocking signal can be applied consecutively, simultaneously, and/or at overlapping times to treat, alleviate, and/or prevent at least a portion of a physiological condition (e.g., at least one abnormal physiological parameter). In some instances, the stimulation signal can at least partially stimulate conduction of at least one afferent fiber and conduction of at least one efferent fiber in the nerve and the blocking signal at least partially block conduction of at least one afferent fiber (which may or may not be the same) and/or at least another efferent fiber in the nerve (which may or may not be/include the at least one efferent fiber). The stimulation signal can activate the parasympathetic response and the blocking signal can attenuate the parasympathetic response. In some other instances, this may be reversed. At 78, the stimulation signal and/or the blocking signal can be adjusted manually and/or with closed loop feedback as described in methods 800 and 900 to treat, alleviate, and/or prevent a portion of a physiological condition (e.g., at least one abnormal physiological parameter).

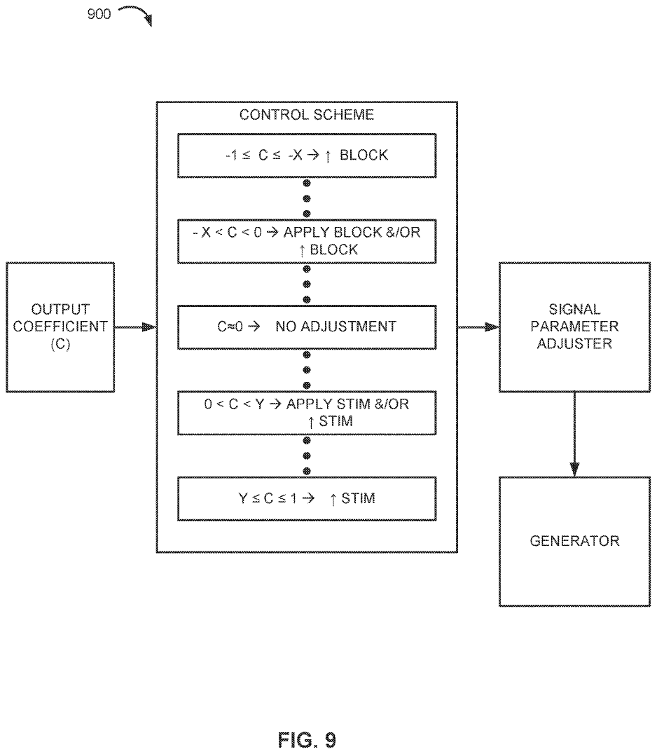

Referring now to FIG. 8, illustrated is a method 800 for closed loop feedback multimodal neuromodulation in the form of bi-polar neuromodulation of a nerve of the autonomic nervous system. At 82, a recording of at least one physiological parameter related to a physiological condition can be received from at least one sensor by a system including at least a processor (like processor 20). At. 84, one or more statistical values of the at least one physiological parameter can be calculated. For example, at least one of a median, a rate of change, an error, or the like can be calculated. As used below, an error from a median physiological parameter and the slope of that error are used for further calculations. At 86, an output coefficient representative of a desired change in the at least one physiological parameter can be calculated based on the one or more statistical values using a control system. The control system can be a fuzzy logic system as described in more detail in the method 900 of FIG. 9. At 88, whether a stimulation signal and/or a blocking signal is applied and/or at least one signal parameter of a stimulation signal and/or at least another signal parameter of a blocking signal can be adjusted based on the output coefficient (also as described in further detail in the method 900 of FIG. 9). In brief when the output coefficient is positive, the stimulation signal can be applied and/or at least one signal parameter of the stimulation signal can be increased; when the output coefficient is zero, the at least one signal parameter of the stimulation signal and the at least the other signal parameter of the blocking signal can be left alone; and when the output coefficient is negative, the blocking signal can be applied and/or at least the other signal parameter of the blocking signal can be increased. At 90, the adjusted at least one signal parameter of the stimulation signal and/or the at least the other signal parameter of the blocking signal can be output to a generator to apply an adjusted signal to change the at least one physiological parameter. If the signal being applied needs to be changed the parameters for the signal that needs to be applied can be output to the generator.

Referring now to FIG. 9, illustrated is a method 900 for implementing a fuzzy loop control system for multi-modal neuromodulation of a nerve. The method 900 can take and generate a coefficient (c), compare the output coefficient with a control scheme for the physiological parameter, and then determine how to adjust one or more parameters of the stimulating and/or blocking signal to cause a desired change to the physiological parameter.

As discussed previously in method 800 a physiological parameter can be recorded over time at a chosen frequency and the recording can be received by a controller including the fuzzy logic control system. A median of the physiological parameter of the patient at a time can be calculated based on the recorded physiological parameter and a physiological parameter error compared to a setpoint and a rate of change of the physiological parameter error can also be calculated. An output coefficient representative of a desired change in the median physiological parameter error and/or a trend in the physiological parameter can be calculated using a fuzzy logic controller. One or more parameters of the blocking signal and/or the stimulation signal can be adjusted based on the output coefficient.

As an example, to calculate the median physiological parameter of the patient at a time, a sensor can be sampled at a frequency in time segments (e.g., 1 kHz in 0.5 second segments). Each of the segments can be loaded into a buffer such that the most recent time period of segments (e.g., 1 second, 2 seconds, 3 seconds, 5 seconds, or the like) are retained and the oldest segment(s) are removed. The segments of the recorded physiological parameter in the buffer can be used for the calculation of the physiological parameter, if required. For example, heart rate can be calculated by taking the mean of the inter-pulse times between successive QRS complex peaks. If the heart rate is outside of an expected value (e.g., outside 75 bpm-400 bpm), then the controller can deem an error occurred (e.g., due to electrical interference on the ECG signal) and the last known good heart rate calculation can be used instead. The same is true for any error due to electrical interference and known expected values. The median of the last predetermined number of physiological parameter and/or physiological parameter calculations (e.g., the last four physiological parameter calculations determined from the last 4 segments saved within the 2 second buffer, or the like) can be used as input for further calculations. By taking the median physiological parameter over the time period a smoothed average physiological parameter can be obtained that can be unaffected, or less affected, by momentary changes. A physiological parameter error can be calculated based on the difference between a predetermined physiological parameter setpoint and the median physiological parameter at the time. A physiological parameter slope (or rate of change of the physiological parameter error) can be calculated based on the most recent median physiological parameter minus the previous median physiological parameter that was calculated.

The fuzzy logic control system can include, for example, four problem “zones”, that each respond to a combination of high/low physiological parameter error and increasing/decreasing physiological parameter slope and a “good” fifth zone. The four problem zones can include: (1) high physiological parameter that is increasing, (2) high physiological parameter that is decreasing, (3) low physiological parameter that is increasing, and (4) low physiological parameter that is decreasing. When the heart rate is deemed to be “good” (e.g., the output coefficient is within a predetermined range around 0 considered to be good for the patient and/or the physiological condition) then the control system can dampen any adjustments and/or not make adjustments to the stimulating and/or blocking signal parameters. For example, if the physiological parameter is heart rate, then with these damping effects, any heart rate with 5 bpm from a “good” setpoint would elicit little or no change to the signal parameters of the stimulating and/or blocking signals, but changes would increase if the heart rate error increased. It should be noted that the fuzzy logic control system can produce/utilize output coefficients all along a gradient of high/low physiological parameter rate error and high/low physiological parameter slope and only some example general guidelines are described below. It should be noted that any increases and/or decreases in signal parameters can be related to how close the output coefficient is to −1, 0, or 1.

If the output coefficient is a large positive value (e.g., within a first predetermined value and 1, the zone (1) described above) then the feedback indicates the physiological parameter error is high and the slope of the physiological parameter error is high, which means the physiological parameter is high and trending higher and needs to be significantly lowered. The signal parameter adjuster can output signal parameters to output a command to generate a stimulation signal (if one is not already being generated/applied), increase the frequency of the stimulating waveform to increase the parasympathetic response, and/or lower the amount of block (e.g., lower the amplitude of the block waveform) to decrease the parasympathetic response. FIG. 9 shows the example of where the stimulation signal (e.g., one or more parameters of the stimulation signal) is increased if the output coefficient is between the first predetermined value and 1. When the output coefficient is positive but between the first predetermined value and about 0 (e.g., the thresholds around 0 considered “normal” for the patient”) (e.g., in the zone (2) described above) then the feedback indicates the physiological parameter error is high, but the slope of physiological parameter error is decreasing. The signal parameter adjuster can output signal parameters that can output a command to generate a stimulation signal (if one is not already being generated/applied), increase the frequency of the stimulating waveform to slightly increase the parasympathetic response and lower the amount of block (e.g., lower the amplitude of the block waveform) to slightly decrease the parasympathetic response, to a lesser amount than if in zone (1), and/or can only output signal parameters that can increase the frequency of the stimulating waveform to increase the parasympathetic response. FIG. 9 shows the example of where the stimulation signal is applied and/or the stimulation signal (e.g., one or more parameters of the stimulation signal) is increased if the output coefficient is between the first predetermined value and about 0.

When the output coefficient is a negative value between about 0 (e.g., the thresholds around 0 considered “normal” for the patient”) and a second predetermined threshold (e.g., in zone (3)) indicating that the physiological parameter is low but increasing. Then, the signal parameter adjuster can output signal parameters that can output a command to generate a blocking signal (if one is not already being generated/applied), increase the blocking signal (e.g., increase the amplitude of the block waveform, but less so than for zone (4) described below) to increase the sympathetic response and/or attenuate the parasympathetic response and/or increase the blocking signal (e.g., increase the amplitude of the block waveform, even less than if only the block is increased) to slightly decrease the parasympathetic response and decrease the frequency of the stimulating waveform to slightly decrease the parasympathetic response. FIG. 9 shows the example of where the blocking signal is applied and/or the blocking signal (e.g., one or more parameters of the blocking signal) is increased if the output coefficient is between the second predetermined value and about 0. When the output coefficient is near negative 1 (e.g., between negative 1 and the second predetermined threshold) indicating the physiological parameter is low and trending lower (e.g., in zone (4). Then, the signal parameter adjuster can output a command to generate a blocking signal (if one is not already being generated/applied), output signal parameters that can increase the blocking signal (e.g., increase the amplitude of the block waveform) to attenuate the parasympathetic response and decrease the frequency of the stimulating waveform to decrease the parasympathetic response. FIG. 9 shows the example where the blocking signal (e.g., one or more parameters of the blocking signal) is increased if the output coefficient is between the second predetermined value and −1. These adjustments to the physiological parameter can continuously update to provide the patient with as normal a physiological parameter as possible (e.g., within predetermined thresholds). The adjusted signal parameters can then be sent to a generator to apply the adjusted signal(s).

### V. Experimental

The following example is shown for the purpose of illustration only and is not intended to limit the scope of the appended claims. The following experiment demonstrates multi-modal, electrical neuromodulation of the vagus nerve using a single implanted bi-polar electrode employing this electrical neuromodulation that can adapt to changes in autonomic state in real-time to modulate heart rate. The use of electrical current to modulate neurons for autonomic regulation requires the ability to both up-regulate and down-regulate vagus activity to reach a desired setpoint.

The electrical neuromodulation uses both stimulation and block of autonomic nerves. Frequency modulation of the stimulation waveform and amplitude modulation of the blocking waveform were each able to successfully titrate the effects on heart rate that they produced. While the FLC controller created was simple, it showed that translation from observed physiological effects to a control scheme can be achieved quite easily and robustly. An FLC is also easily expandable to include other inputs and outputs; plans to incorporate additional measurements such as blood pressure, heart rate variability (HRV), and neurochemical measurements are underway, and can be added to the controller by simply creating new rules. Additional output parameters may also be included, such as placing independent cuffs, or in some instances additional contacts, on the vagus on both sides, modulating the amplitude or pulse width of the stimulation or placing electrodes on cardiac sympathetic nerves.

### I. Multi-Modal Neuromodulation Introduction

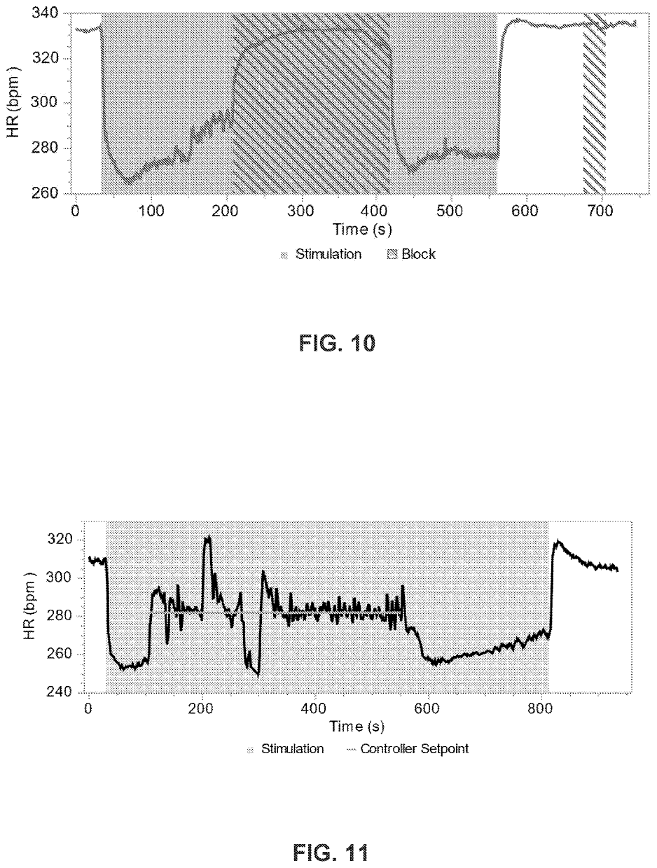

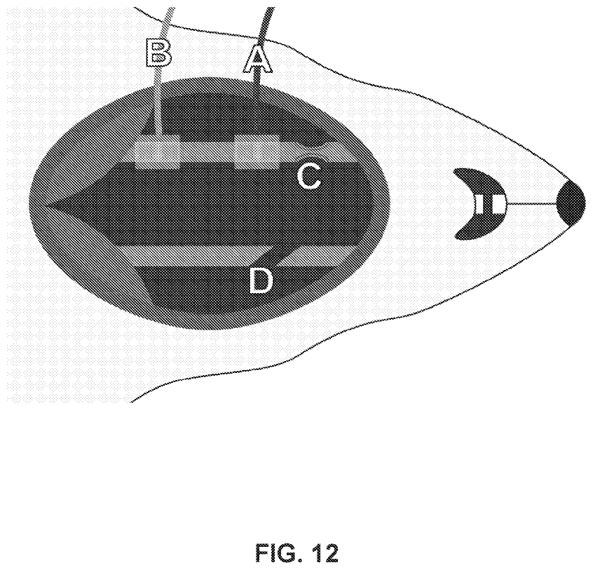

Multi-modal neuromodulation of the autonomic nervous system can include stimulation and block. Stimulation of autonomic nerves can be done at frequencies in the range 1-30 Hz to increase neural activity. Vagus nerve stimulation (VNS) has been shown to be sensitive to frequency adjustments, which can be used to control the effect more precisely as compared to amplitude modulation. Kilohertz frequency alternating current (KHFAC) is a proven technique for blocking action potential conduction to reduce neural activity. Additionally, KHFAC can be reliably modulated by amplitude modulation. Although there are many types of commonly used closed-loop controllers, many conventional methods do not respond well to long system delays or discontinuities. Fuzzy logic control (FLC) is a state-based controller that can describe the discontinuities of the system linguistically and then translate the state transition to a continuous output signal. In this preparation, a single bipolar electrode was placed on the vagus nerve and controlled by a fuzzy logic controller to deliver both stimulation and KHFAC to control heart rate. The FLC was able to both change the heart rate to selected values and maintain the heart rate at a constant value in response to a physiological perturbation. Results of an example early trial (also using the step shown in FIG. 12) can be seen in FIG. 10. The plot of FIG. 10 shows an in vivo example of the response of a rat's heart rate to 30 Hz stimulation and subsequent constant 10 kHz block of the cervical vagus nerve distal to the stimulation. Notice that even at a steady input (or no input at all), heart rate can fluctuate significantly. Note that the block by itself at the end has no effect.

In most applications, neuromodulation intended to activate a nerve is modulated by adjusting the amplitude of the signal. The autonomic effects of stimulation frequency are more complex and dependent on the interactions of the target nerve with the rest of the system. In mixed nerves, such as the vagus, both afferent and efferent pathways may be stimulated. In a recent study of VNS, waveforms with different frequencies, amplitudes and pulse widths were tested to determine the combination that produced the lowest amount of off target effects during VNS. The responses to changes in each of these parameters form the neural fulcrum. This study demonstrated the importance of exploring the whole parameter space when using electrical stimulation in autonomic targets. The results from this study show that while it is possible to achieve a gradation of the effect from stimulation using amplitude modulation, the changes from frequency modulation were easier to predict and control. In general, amplitude modulation of a waveform effects how many individual nerve fibers in a nerve tract are activated, as some fibers require more current than others to activate (small fibers and fibers deeper within the nerve tract require more current). Frequency modulation at the high amplitudes used in this application activates all fibers but varies how often the fibers are activated. Each approach has its advantages and disadvantages, but for this system, frequency modulation was chosen.

Autonomic systems can be difficult to control due to long time delays and discontinuities. In the case of the cardiovascular system, the chain of mechanisms by which neural activity controls cardiac activity each have their own time courses and delays, and these effects change over time. For example, the vagus nerve controls heart rate by releasing acetylcholine into the heart muscle from the endings of each of the individual nerve fibers. The rate at which acetylcholine is released from the nerve, the rate that it diffuses across the gap between the nerve and cardiac cells, the rate that it binds to the cell receptors, and the amount of change that this caused to heart rate can all vary significantly based on the specific physiological conditions it is in. Since all the variables that control these responses cannot be known (both the dynamics of vagus stimulation and block on direct cardiac outputs, as well as the internal compensatory mechanisms of the animal), conventional controllers such as proportional, integral, derivative (PID) controllers are not well-suited to handle these systems. Aside from the differences over time in in any single patient, physiological systems may exhibit widely different properties between patients. A PID controller had been attempted in preliminary experiments using DC nerve block but became unstable due to the widely variable delays seen in the system, and hysteresis that was seen especially during the blocking portions. A good balance between stability and responsiveness was difficult to find for an individual rat, with animal-to-animal variations being even harder to control for. An example of one of these trials can be seen in FIG. 11. The plot of FIG. 11 shows the heart rate of a rat attempting to be controlled using DC nerve block via amplitude modulation. Note the large excursions around 200-300 seconds. The controller is tuned for stability for this individual animal, but the PID system is unable to respond to random large, unexpected changes in heart rate caused by the rat's internal compensatory mechanisms, even when the vagus stimulation remains at a constant value.

A Fuzzy Logic Controller (FLC) is an adaptive controller that provides an intuitive mapping of descriptive properties of systems to controller output, and it has been shown to perform well as a simple control scheme when a plurality of physiological variables are not known. In this study, an FLC was used to merge a KHFAC block and stimulation paradigms into a single controller of heart rate by modulating the electrical activity of the vagus nerve.

### II. Methods

### A. Surgical Preparation and Electrodes

An acute experiment was performed on an adult male Sprague-Dawley rat weighing 486 g. Under institutional approval, the animal was anesthetized using inhaled Isoflurane (5% induction, 2% maintenance), and intubated with a 14 ga angiocatheter and ventilated at 60 breaths per minute. Intubation and ventilation were performed to mitigate the effects on breathing elicited by cervical VNS. The rat neck was then dissected to expose the cervical vagus nerve on both sides. Two bipolar electrodes were placed on the right vagus, approximately 5 mm apart edge-to-edge. The electrodes each had two platinum contacts 1 mm by 1 mm, 2 mm apart center-to-center. The left vagus was cut, and the right vagus was crushed with forceps at the rostral end of the dissection, proximal to both electrodes; this was done to remove the tonic vagal tone and limit the effects from afferent action potentials. The surgical setup is illustrated in FIG. 12. The more proximal electrode was used to apply stimulation to simulate a varying vagal tone (“stimulation electrode,” labeled as A in FIG. 12), and the distal of the two electrodes was used to apply the controller output (“control electrode”, labeled as B in FIG. 12). The labels ‘C’ and ‘D’ show the crushing of the proximal right-side vagus and cutting of the left-side, respectively. In a clinical application, only the control electrode would be present; the stimulation electrode is only used to imitate a diseased state in this preparation. Proximal stimulation was provided by an S88 stimulator through an isolated voltage-to-current transformer (Grass Technologies, West Warwick, RI, USA). Distal stimulation was provided by a Caputron voltage-to-current stimulator (Hillsborough NJ, USA), driven by a NI-USB 6259 BNC data acquisition and output device (National Instruments, Austin, TX, USA).

A three-lead ECG was also recorded from needle electrodes, one in each arm and another in the abdomen. These were fed into a CED 1902 amplifier and Power1401-3 DAQ and analyzed in Spike2 software (all CED, Cambridge Electronic Design, Cambridge, EN, UK). The ECG signal was also fed into the NI DAQ device for use in heart rate calculation for the controller.

### B. Controller Design

The experiments were performed using a fuzzy logic controller (FLC) that was programmed in National Instruments' LabVIEW software. The controller software would first calculate heart rate using the measured ECG signal. The ECG was sampled at 1 KHz in 0.5 second segments. These segments were loaded into a buffer, such that the last 2 seconds of data were retained. The 2 second buffer was then used to calculate heart rate by taking the mean of the inter-pulse times between successive QRS complex peaks (the largest deflection of an EKG signal). If the heart rate was outside of expected values (75 bpm-400 bpm), this was deemed an error, generally due to electrical interference on the ECG signal. If an error was detected here, the last know good heart rate calculation was used.

The median of the last 4 heart rate calculations (again, the last 2 seconds) was used as the heart rate for the resultant output calculation. Taking the median heart rate over two seconds using these two methods allowed a smoothed average heart rate that is unaffected by momentary changes in heart rate (premature ventricular contractions, sinus arrythmia from breathing) to be obtained, so that the measurements (and therefore output waveforms) remain relatively smooth. The heart rate error was defined as the difference between the heart rate setpoint and this median heart rate. The heart rate slope was the most recent median heart rate minus the previous median heart rate (0.5 seconds before).

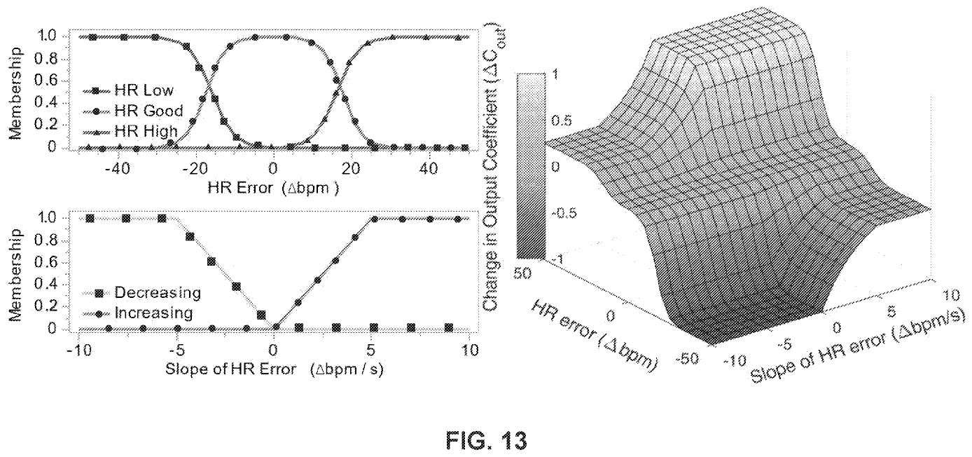

After evaluating the heart rate error and the heart rate error's rate of change, the controller outputs a desired change in the output coefficient (cₒᵤₜ). This metric is used to determine which therapeutic parameters to use, with positive values corresponding with low frequency, activating stimulation, and negative values corresponding to KHFAC blocking waveforms. The larger the absolute value, the stronger the effect becomes (i.e., an increasing positive value will increase the frequency of the activating stimulation, and a decreasing negative value will increase the blocking waveform amplitude, see FIG. 13). The controller outputs were normalized to the refresh rate and an additional gain was applied, such that at full power, the maximum change in heart rate would be 5 bpm over a 1 second period. In FIG. 13, the left graphs show the membership functions for the fuzzy logic controller. The right graph shows the resulting FLC input-output map in detail. Note the smaller changes in Cout as the heart rate error gets smaller, and when the heart rate is trending in the direction of the setpoint.

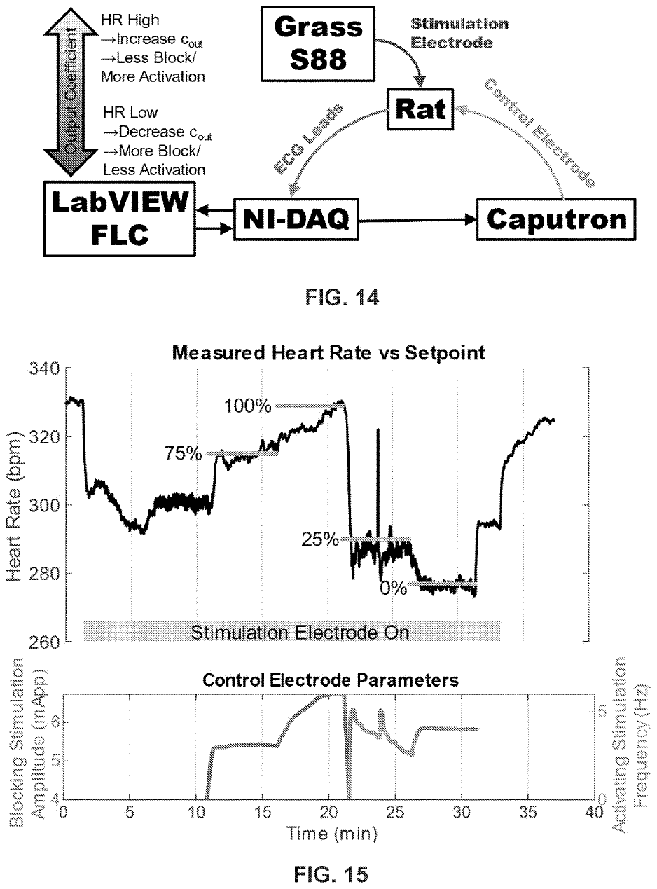

The software calculates Cout using an FLC that takes the heart rate error and slope. The fuzzy system designed was based on preliminary testing of both the KHFAC waveform and stimulation parameters. Fuzzy logic was chosen for this project for its ease in translating our observations into controller parameters. The FLC was designed to have 4 “zones,” with each responding to a combination of high/low heart rate, and an increasing/decreasing heart rate. When the heart rate was deemed to be in the “good,” (i.e., heart rate error was small), the controller was designed to severely dampen any changes, so as not to overshoot the target heart rate. With these damping effects, any heart rate within ±5 bpm of the setpoint would see little change in the output parameters, with changes increasing as the heat rate error increased. FIG. 14 shows a schematic for the hardware used to implement this controller. The diagram of FIG. 14 shows the flow of data for the controller. The ECG leads feed into the NI-DAQ, which is used by the LabVIEW application to calculate the heart rate error and change in heart rate. The software then outputs the stimulation waveform from the NI-DAQ into a Caputron voltage-to-current converter, which drives current through the control electrode. The Grass S88 stimulator is also used to supply synthetic vagal tone to the system. The upper left part of the figure shows the correlation between Cout and the stimulation parameters.

C. Testing Protocols

### 1) Calibration

Before the controller was implemented, the responses of the rat to both activation and blocking waveform were assessed. The value of Cout was designed to be approximately equal change in heart rate (ex., if cₒᵤₜ=12, Δbpm≈12). For this to work in practice, sensitivities for both stimulation and KHFAC needed to be found. For the calibration of stimulation, the saturation amplitude was first found with 30 Hz, 100 μs biphasic pulses (50 μs per phase) delivered through the control electrode. This was done by increasing the amplitude of stimulation and watching the heart rate response drop; that level at which increasing the amplitude no longer decreased the heart rate further was declared to be the saturation amplitude. This amplitude was then used for a 1 Hz stimulation, where the heart rate relative to baseline was recorded. This was repeated in 1 Hz steps up to 12 Hz, where the stimulation effect was seen to top out. These points were then plotted, and a regression line was fitted to give a basic fit of the response.

A similar calibration was done for the blocking waveform. First, a saturation threshold was found for the stimulation electrode at 30 Hz, 50 μs monophasic pulses. This was done in a similar manner as above. The control electrode then output a high-amplitude 10 kHz square wave, to completely block the conduction of action potentials. This amplitude was then lowered until decreases in the heart rate were seen. The amplitude was lowered in 1 mApp steps until the heart rate was no longer decreasing. The points between the lowest amplitude that completely blocked and the lowest amplitude that produced any block were then plotted, and a regression line was fit to them. The controller was designed to begin applying block at the lowest amplitude at which any block was seen, to avoid a sub-block zone that has been seen to lead to onset activity.

### 2) Experiment 1: Same Stimulation, Different Setpoints

The first test performed was designed to test how well the controller could control the heart rate to a range of values under static conditions. The first step was setting the stimulation electrode to a non-saturation level. This was done by turning on the stimulation at the saturation amplitude at 30 Hz, and then lowering the frequency until 50% of the change in heart rate was seen. Using this style of notation, the heart rates were normalized so that the baseline HR without any stimulation was 100%, and the lowest HR achieved with VNS was 0%. The heart rate was allowed to settle around 50% before the controller was turned on. After settling, the HR setpoint on the controller was set to the 75%, 100%, 25%, and then 0% for 5 minutes each. The controller would adjust the output of the control electrode to modulate vagal activity to reach these setpoints.

### 3) Experiment 2: Varying Stimulation, Same Setpoint

The second type of experiment performed involved keeping the HR setpoint at a static value and modulating the synthetic vagal tone from the stimulation electrode. This was performed by setting the controller setpoint to the 50% HR level, and then increasing and decreasing the stimulation electrode frequency, and allowing the controller to compensate. This test was designed to mimic a clinical application where a constant heart rate is desired, but vagal activity is changing, such as in an episode of vasovagal syncope.

### III. Results

### A. Calibration

The calibration data collected is used in the scaling of the controller outputs, to try and roughly match the output coefficient to the change in HR. The activation plot was very linear and with a moderate degree of sensitivity at −5.25 bpm/Hz, while the blocking slope was very steep at 39 bpm/mApp.

### B. Experiment 1: Same Stimulation, Different Setpoints

An example trial for this experiment type can be seen in FIG. 15. In FIG. 15, the top plot shows the measured heart rate, and the heart rate setpoints throughout the trial. The gold bar across the bottom indicates when the stimulating electrode is turned on. The bottom plot shows the controller outputs, both the blocking amplitude (blue, left side) and the activating stimulation frequency (orange, right side). In this trial, the controller starts by using blocking waveform, as indicated by the blue line on the bottom graph to raise the heart rate to the 75% level. The block amplitude stabilizes at around 5.4 mApp after taking about 1 minute to reach the setpoint from the 50% base level. After 5 minutes, the setpoint was moved to the 100% level, taking about 4 minutes to reach with an amplitude of about 6.7 mApp. Going from the 100% level to the 25% level, the controller quickly moves from providing a 10 kHz blocking waveform to a low frequency stimulation. The controller hits the setpoint in about 1 minute and does not settle to a specific value within the 5-minute window; the stimulation frequency at the end of the time frame was around 2.5 Hz. Transitioning to the 0% HR level, the controller again takes about 1 minute, and settles near 4.0 Hz. After this setpoint, the controller was turned off, and the heart rate returned to HR level near 35%, lower than the initial 50% synthetic vagal tone level. After the synthetic tone was turned off, the baseline heart rate also appeared to have dropped by around 5 bpm.

### C. Experiment 2: Varying Stimulation, Same Setpoint

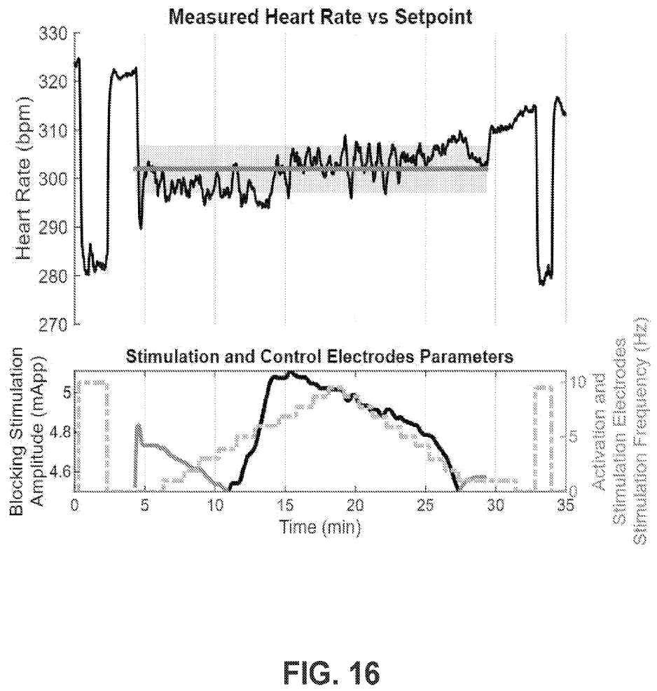

In the trial shown in FIG. 16, the effectiveness of the controller at maintaining a static setpoint under varying conditions is examined. The top plot shows the measured heart rate and the 50% setpoint for the controller. The green line shows the setpoint, and the lighter green rectangle shows the setpoint +or −5 bpm. The bottom graph shows the controller stimulation outputs for both blocking amplitude (blue, left) and activating stimulation frequency (orange, right). The frequency of the synthetic vagal tone is shown in the gold line, also on the right-hand axis. The beginning and ends of the trial show the baseline 100% HR level, and the 0% maximal VNS level for reference. The setpoint was at the 50% HR value for the duration of the trial. After turning the controller on, it reached the setpoint via activating stimulation and settled in about 1 minute. After settling, the stimulating electrode was turned on at 1 Hz. The frequency of the stimulating electrode's synthetic vagal tone was increased roughly every minute by 1 Hz up to 10 Hz; the frequency was then dropped back down to 1 Hz. The graph shows low vagal tone requiring activating stimulation to reach the setpoint, and high vagal tones requiring conduction block.

From the above description, those skilled in the art will perceive improvements, changes, and modifications. Such improvements, changes and modifications are within the skill of one in the art and are intended to be covered by the appended claims.

1. A system comprising:
an electrode configured to be in communication with at least one nerve of the autonomic nervous system to affect a physiological condition via multimodal neuromodulation, wherein the electrode is configured to apply a neuromodulation signal to modulate at least one physiological parameter related to the physiological condition at a time, wherein the neuromodulation signal comprises at least one signal parameter and to apply another neuromodulation signal to modulate the at least one physiological parameter related to the physiological condition at another time, wherein the other neuromodulation signal comprises at least another signal parameter; and
a generator configured to generate and provide the neuromodulation signal, comprising the at least one signal parameter, to the electrode and the other neuromodulation signal, comprising the at least the other signal parameter, to the electrode to continuously adjust to changes in the at least one physiological parameter due to the physiological condition.

2. The system of claim 1, wherein the neuromodulation signal is configured to upregulate the at least one physiological parameter and the other neuromodulation signal is configured to downregulate the at least one physiological parameter.

3. The system of claim 2, wherein the neuromodulation signal is configured to activate a parasympathetic response and the other neuromodulation signal is configured to attenuate the parasympathetic response.

4. The system of claim 1, wherein the physiological condition comprises a cardiovascular disease and the at least one physiological parameter is a heart rate.

5. The system of claim 1, wherein the at least one nerve of the autonomic nervous system is a at least one vagus nerve.

6. The system of claim 1, further comprising a controller comprising a memory storing instructions and a processor configured to execute the instructions to:
receive at least one recording of the at least one physiological parameter;
calculate a median and an error of the at least one physiological parameter at a time based on the at least one recording of the at least one physiological parameter;
calculate an output coefficient representative of a desired change in the at least one physiological parameter using a control system based at least in part on the median and the error;
determine whether the neuromodulation signal or the other neuromodulation signal should be applied based on the output coefficient, wherein the neuromodulation signal or the other neuromodulation signal is applied based on the output coefficient; and
adjust the at least one signal parameter of the neuromodulation signal or the at least the other signal parameter of the other neuromodulation signal based on the output coefficient.

7. The system of claim 6, wherein the output coefficient is a value between −1 and 1, and the adjustment is such that:
when the output coefficient is positive, the neuromodulation signal is provided to the electrode and/or the at least one signal parameter of the neuromodulation signal is increased;
when the output coefficient is zero, no action is taken; and
when the output coefficient is negative, the other neuromodulation signal is provided to the electrode and/or the at least the other signal parameter of the other neuromodulation signal is increased.

8. The system of claim 7, wherein when the output coefficient is positive and above a threshold then, the at least one signal parameter of the neuromodulation signal is increased and when the output coefficient is negative and below another threshold, then the at least the other signal parameter of the other neuromodulation signal is increased.

9. The system of claim 6, wherein the controller comprises the control system, wherein the control system is a fuzzy logic control system.

10. The system of claim 6, further comprising at least one sensor configured to record the at least one physiological parameter and send the recording of the at least one physiological parameter to the controller.

11. The system of claim 1, wherein the neuromodulation signal comprises a stimulation current and the other neuromodulation signal comprises a kilohertz frequency alternating current (KHFAC) block.

12. A method for using an electrode for bi-modal neuromodulation of at least one nerve of the autonomic nervous system, wherein the method comprises:
receiving, by a system comprising a processor, a recording of at least one physiological parameter related to a physiological condition from at least one sensor;
calculating, by the system, one or more statistical values of the at least one physiological parameter;
calculating, by the system, an output coefficient representative of a desired change in the at least one physiological parameter based on the one or more statistical values using a control system;
adjusting, by the system, at least one signal parameter of a stimulation signal and/or at least another signal parameter of a blocking signal based on the output coefficient; and
outputting, by the system, the adjusted at least one signal parameter of the stimulation signal and/or the at least the other signal parameter of the blocking signal to a generator,
wherein the generator provides the blocking signal to the electrode at a time and the stimulation signal to the electrode at another time such that the blocking signal and the stimulation signal are applied to the at least one nerve of the autonomic nervous system to alter the at least one physiological parameter to continuously adjust to changes in the at least one physiological parameter due to the physiological condition.

13. The method of claim 12, further comprising
generating, by the generator in communication with the system, the stimulation signal having the at least one signal parameter and the blocking signal have the at least the other signal parameter;
applying, by the electrode, the stimulation signal having the at least one signal parameter to the at least one nerve of the autonomic nervous system; and
applying, by the electrode, the blocking signal having the at least the other signal parameter to the at least one nerve of the autonomic nervous system.

14. The method of claim 12, wherein the stimulation signal stimulates conduction of at least one afferent fiber and conduction of at least one efferent fiber in the nerve and the blocking signal at least partially blocks conduction of at least another efferent fiber in the nerve.

15. The method of claim 12, wherein the adjusting further comprises:
when the output coefficient is positive:
applying, by the system, the stimulation signal and/or
increasing, by the system, the at least one signal parameter of the stimulation signal;
when the output coefficient is zero, not adjusting, by the system, the at least one signal parameter of the stimulation signal or the at least the other signal parameter of the blocking signal; and
when the output coefficient is negative:
applying, by the system, the blocking signal and/or
increasing, by the system, the at least the other signal parameter of the blocking signal.

16. The method of claim 15, further comprising:
when the output coefficient is positive and above a threshold, increasing, by the system, the at least one signal parameter of the stimulation signal; and
when the output coefficient is negative and below another threshold, increasing, by the system, the at least the other signal parameter of the blocking signal.

17. The method of claim 12, wherein the stimulation signal comprises a stimulation current and the blocking signal comprises a kilohertz frequency alternating current (KHFAC) block.

18. The method of claim 12, wherein the stimulation signal is configured to activate a parasympathetic response and the blocking signal is configured to attenuate the parasympathetic response.

19. The method of claim 12, wherein the physiological condition comprises a cardiovascular disease, the at least one physiological parameter is a heart rate, and the at least one nerve of the autonomic nervous system is at least one vagus nerve.

20. A system comprising:
a generator configured to generate at least a blocking signal and a stimulation signal;
a bi-polar electrode configured to be positioned in electrical communication with a vagus nerve of a patient, the bi-polar electrode comprising:
a contact configured to apply the stimulation signal to the vagus nerve to activate parasympathetic neural activity and to apply the blocking signal to the vagus nerve to block parasympathetic neural activity; and
a return contact;
at least one sensor configured to record an electrocardiogram (ECG) of the patient;
a controller in communication with the generator and the at least one sensor, the controller comprising a non-transitory memory configured to store instructions and processor configured to execute the instructions to:
calculate a median heart rate error of the patient at a time based on the ECG and a heart rate error slope,
calculate an output coefficient representative of a desired change in the median heart rate error and/or the heart rate error slope using a fuzzy logic controller, and
adjust whether the stimulation signal or the blocking signal is applied and/or one or more parameters of the blocking signal or the stimulation signal based on the output coefficient.
